(19) United States
(12) Patent Application Publication
(10) Pub. No.: US 2024/0062872 A1
(43) Pub. Date: Feb. 22, 2024

(54) COHORT SLEEP PERFORMANCE EVALUATION

(71) Applicant: ResMed Sensor Technologies Limited, Dublin (IE)

(72) Inventors: Luca CERINA, Dublin (IE); Jessica Xu, Dublin (IE); Varunl Lakshana Vithanage Femando, Dublin (IE); Aolbhe Tumer, Dublin (IE)

(21) Appl. No.: 18/267,941

(22) PCT Filed: Dec. 16, 2021
(86) PCT No.: PCT/IB2021/061883

§ 371 Date: Jun. 16, 2023

Related U.S. Application Data
(60) Provisional application No. 63/127,597, filed on Dec. 18, 2020.

(51) Int. Cl. G16H 20/40; A61M 16/00; G16H 40/67; G16H 10/20; G16H 50/50

(52) U.S. Cl. CPC G16H 20/40; A61M 16/024; A61M 16/0003; G16H 40/67; G16H 10/20; G16H 50/50

(57) ABSTRACT

Certain aspects and features of the present disclosure relate to evaluating the sleep performance of a cohort of multiple individuals sleeping in a shared environment (e.g., a single bed, a single room, a set of adjacent rooms, or a single household). Individual or concerted sleep performance scores can be determined, as well as other sleep performance metrics. The evaluation of sleep performance for the entire cohort can be useful when one of the individuals is being treated (e.g., with a respiratory therapy device) for a sleep-related and/or respiratory disorder. Evaluation of the cohort can help identify actions that can be taken to improve the sleep performance of all individuals in the cohort. In some cases, parameters of a user's therapy device are adjusted based on the monitored sleep performance of another individual in the cohort.

### CROSS REFERENCE TO RELATED APPLICATIONS

The present application claims the benefit of U.S. Provisional Patent Application No. 63/127,597, filed Dec. 18, 2020, and entitled “COHORT SLEEP PERFORMANCE EVALUATION,” the disclosure of which is hereby incorporated by reference in its entirety.

### TECHNICAL FIELD

The present disclosure relates to treatment of sleep conditions generally and more specifically to monitoring sleep performance in a multi-person environment.

### BACKGROUND

Many individuals suffer from sleep-related and/or respiratory disorders such as, for example, Periodic Limb Movement Disorder (PLMD), Restless Leg Syndrome (RLS), Sleep-Disordered Breathing (SDB) such as Obstructive Sleep Apnea (OSA), Central Sleep Apnea (CSA), other types of apneas such as mixed apneas and hypopneas, Respiratory Effort Related Arousal (RERA), Cheyne-Stokes Respiration (CSR), respiratory insufficiency, Obesity Hyperventilation Syndrome (OHS), Chronic Obstructive Pulmonary Disease (COPD), Neuromuscular Disease (NMD), chest wall disorders, and insomnia. The sleep-related respiratory disorders can be associated with one or more events that may occur during sleep, such as, for example, snoring, an apnea, a hypopnea, a restless leg, a sleeping disorder, choking, an increased heart rate, labored breathing, an asthma attack, an epileptic episode, a seizure, or any combination thereof. Individuals suffering from such sleep-related respiratory disorders are often treated using one or more medical devices to improve sleep and reduce the likelihood of events occurring during sleep. An example of such a device is a respiratory therapy system that can provide positive airway pressure to the individual, although other devices may be used.

Additionally, many individuals suffering from sleep-related and/or respiratory disorders sleep in the same environment as one or more other individuals. Any individual in an environment can affect the sleep performance of another individual in the environment. Examples include disruptions due to movement or noise (e.g., from a first bed partner entering a bed after the second bed partner is in initial stages of sleep), disruptions due to common sleep hygiene (e.g., the timing of using digital devices or eating meals immediately prior to sleep), or other factors. Not only can the treatment of an individual suffering from sleep-related and/or respiratory disorders affect a healthy individual's sleep performance, but a healthy individual's actions can affect the sleep performance of the individual suffering from sleep-related and/or respiratory disorders.

There is a need to provide meaningful metrics regarding the sleep performance of individuals in environments that include one or more other individuals. Such meaningful metrics can be used to improve the overall sleep quality of one or more of the individuals in the environment, such as to improve compliance and user engagement with a sleep therapy, identify actions that have positive effects on sleep performance, and otherwise improve sleep performance.

### SUMMARY

Certain aspects and features of the present disclosure relate to a method comprising: receiving sensor data from one or more sensors, the sensor data being associated with a sleep session of an individual in an environment; determining first sleep performance data from the sensor data; receiving second sleep performance data, the second sleep performance data being associated with a sleep session of a user of a respiratory therapy device in the environment, the user being different than the individual; generating one or more sleep performance metrics using the first sleep performance data and the second sleep performance data; and presenting the one or more sleep performance metrics.

In some cases, the one or more sleep performance metrics includes i) a concerted sleep performance score; ii) an individual sleep performance score associated with the individual; iii) an individual sleep performance score associated with the user; iv) a hypnogram associated with the individual; v) a hypnogram associated with the user; vi) a therapy score for the user; vii) a resonance score; or viii) any combination of i-vii. In some cases, the sleep session of the individual and the sleep session of the user overlap, fully or partially, in time. In other cases, the sleep session of the individual and the sleep session of the user do not overlap in time, such as when one of the individual or user is a shift worker with unique sleep-wake pattern. In some cases, the first sleep performance data includes sleep stage information or sleep state information; and wherein the second sleep performance data includes respiratory therapy device usage information. In some cases, generating the one or more sleep performance metrics includes generating a concerted sleep performance score, and wherein generating the concerted sleep performance score includes: generating a first sleep performance score using the first sleep performance data; generating a second sleep performance score using the second sleep performance data; and generating the concerted sleep performance score using the first sleep performance score and the second sleep performance score.

In some cases, the method further comprises: receiving goal information associated with the first user and the second user, wherein the goal information is indicative of a goal associated with i) the sleep session of the individual, ii) the sleep session of the user, or iii) a combination of i and ii; generating a goal status update, wherein generating the goal status update includes evaluating the goal information using i) the first sleep performance data; ii) the second sleep performance data; or iii) a combination of i and ii; and outputting the goal status update. In some cases, receiving goal information includes: generating a set of one or more suggested goals; and receiving a selection for a selected goal out of the set of suggested goals. In some cases, generating the set of suggested goals includes: presenting a questionnaire containing one or more questions; receiving response information in response to presenting the questionnaire; and generating the set of suggested goals using the received response information. In some cases, generating the set of suggested goals includes: accessing historical sleep performance data associated with historical sleep performance metrics; identifying one or more factors as influencing the historical sleep performance metrics; determining, for each of the one or more factors, a suggested action estimated to improve a future sleep performance metric; and generating the set of suggested goals using the suggested action for each of the one or more factors. In some cases, generating the set of suggested goals includes: receiving demographic information associated with the individual or the user; and generating the set of suggested goals using the received demographic information. In some cases, generating the set of suggested goals includes: receiving historical respiratory therapy device usage information associated with the user; and generating the set of suggested goals using the received historical respiratory therapy device usage information. In some cases, generating the set of suggested goals includes: receiving subjective feedback associated with a plurality of historical sleep sessions; and generating the set of suggested goals using the subjective feedback. In some cases, evaluating the goal further includes using the sensor data. In some cases, evaluating the goal using the sensor data includes estimating a distance between the individual and the user using the sensor data. In some cases, receiving goal information includes receiving a target completion date associated with the goal, and wherein receiving the target completion data includes automatically determining the target completion date using i) the first sleep performance data; ii) the second sleep performance data; iii) historical sleep performance data; or iv) any combination of i-iii. In some cases, the goal information includes a goal associated with a start time of a future sleep session of the individual and a start time of a future sleep session of the user. In some cases, the goal information includes a goal associated with a distance between the individual and the user at a future sleep session. In some cases, the goal information includes a goal associated with a future use of the respiratory therapy device. In some case, upon completion of a goal, a further selection of goals is suggested. The further selection of goals may be based on one or more of the completed goal, the time taken to achieved the completed goal, sleep performance data and/or subjective data of the user, sleep performance data and/or subjective data of the individual, or a combination thereof.

In some cases, the method further comprises: identifying a coaching suggestion for improving a future sleep performance metric; and providing, after the first sleep session, the coaching suggestion. In some cases, identifying the coaching suggestion includes: receiving subjective feedback associated with a plurality of historical sleep sessions; and generating the coaching suggestion using the subjective feedback. In some cases, identifying the coaching suggestion includes: accessing historical sleep performance data associated with historical sleep performance metrics; identifying one or more factors as influencing the historical sleep performance metrics; determining, for each of the one or more factors, a suggested action estimated to improve a future sleep performance metric; and generating the coaching suggestion using the suggested change for each of the one or more factors.

In some cases, the method further comprises providing an incentive based on the first sleep performance data and the second sleep performance data. In some cases, providing the incentive is further based on a comparison between the one or more sleep performance metrics and a historical sleep performance metric. In some cases, generating the one or more sleep performance metrics includes generating a concerted sleep performance score, and wherein generating the concerted sleep performance score includes: generating a first sleep performance score using the first sleep performance data; generating a second sleep performance score using the second sleep performance data; and generating the concerted sleep performance score using the first sleep performance score and the second sleep performance score; and wherein providing the incentive occurs when the first sleep performance score exceeds a first threshold and the second sleep performance score exceeds a second threshold. In some cases, providing the incentive includes providing a first individual incentive associated with the individual and providing a second individual incentive associated with the user. In some cases, the method further comprises: providing a first individual incentive associated with the individual when the first sleep performance score exceeds the first threshold; and providing a second individual incentive associated with the user when the second sleep performance score exceeds the second threshold. In some cases, the method further comprises: providing a first individual incentive associated with the user when the first sleep performance score exceeds the first threshold; and providing a second individual incentive associated with the individual when the second sleep performance score exceeds the second threshold.

In some cases, the method further comprises: transmitting summary information based on the first sleep performance data, wherein the summary information, when received by a user device associated with the user, is usable to generate an entry on a feed of historical summary information associated with the individual. In some cases, the method further comprises: receiving feedback in response to generation of the entry, wherein the feedback is indicative of a reaction. In some cases, generating the one or more sleep performance metrics includes generating a first sleep performance score using the first sleep performance data, and wherein the summary information includes the first sleep performance score. In some cases, the method further comprises: receiving summary information on a user device associated with the individual, wherein the summary information is based on the second sleep performance data; and generating an entry on a feed of historical summary information associated with the user using the received summary information. In some cases, generating the one or more sleep performance metrics includes generating a second sleep performance score using the second sleep performance data, and wherein the summary information includes the second sleep performance score. In some cases, the second sleep performance data is determined using the sensor data, and wherein the sensor data is further associated with the sleep session of the user in the environment. In some cases, the second sleep performance data is determined using second sensor data from a second set of one or more sensors, the second sensor data being associated with the sleep session of the user in the environment.

Certain aspects and features of the present disclosure relate to a method, comprising: supplying air to a user interface using a respiratory therapy device, the user interface being worn by a user engaging in a sleep session in an environment; receiving sleep session data associated with a sleep session of an individual in the environment, the individual being different than the user; and adjusting a parameter of the respiratory therapy device in response to the received sleep session data. In some cases, adjusting the parameter occurs dynamically during the sleep session of the user and the sleep session of the individual. In some cases, the sleep session data includes sleep stage data of the individual, and wherein adjusting the parameter of the respiratory therapy device is based on the sleep stage data. In some cases, adjusting the parameter of the respiratory therapy device includes: adjusting the parameter to a first setting when the sleep session data is indicative that the individual is awake; and adjusting the parameter to a second setting when the sleep session data is indicative that the individual is asleep, wherein the respiratory therapy device is quieter when the parameter is adjusted to the first setting than when the parameter is adjusted to the second setting.

In some cases, the method further comprises: receiving first sensor data associated with the sleep session of the user; receiving second sensor data associated with the sleep session of the individual, wherein the sleep session data associated with the second sleep session is determined using the second sensor data; and synchronizing the first sensor data and the second sensor data. In some cases, the method further comprises improving a signal-to-noise ratio of a signal of the first sensor data using the synchronized second sensor data. In some cases, the method further comprises: detecting a possible event using the first sensor data; and confirming the event using the synchronized second sensor data. In some cases, the method further comprises estimating a position of the user using the synchronized first sensor data and synchronized second sensor data. In some cases, the method further comprises: establishing a wireless connection with a user device associated with the individual, wherein receiving the sleep session data occurs using the wireless connection; and measuring characteristics of the wireless connection; and determining location information of the individual based on the measured characteristics of the wireless connection. In some cases, the method further comprises adjusting the parameter of the respiratory therapy device based on the location information. In some cases, the wireless connection is a Bluetooth connection.

In some cases, the environment is a building. In some cases, the environment is a pair of adjacent rooms. In some cases, the environment is a room. In some cases, the environment is a sleeping surface.

Certain aspects and features of the present disclosure relate to a method, comprising: generating a simulated respiratory therapy device sound; outputting the simulated respiratory therapy device sound; monitoring the outputted simulated respiratory therapy device sound using a microphone; and adjusting output of the simulated respiratory therapy device sound based on the monitored outputted simulated respiratory therapy device sound.

In some cases, the method further comprises accessing a set of prescribed respiratory therapy settings, wherein generating the simulated respiratory therapy device sound is based on the set of prescribed respiratory therapy settings. In some cases, the method further comprises accessing a set of therapy settings of a respiratory therapy device, wherein generating the simulated respiratory therapy device sound is based on the set of therapy settings of the respiratory therapy device. In some cases, the method further comprises: receiving an adjustment command; adjusting volume of the simulated respiratory therapy device in response to receiving the adjustment command; and providing a respiratory therapy recommendation based on the adjusted volume of the simulated respiratory therapy device. In some cases, the respiratory therapy recommendation includes i) a respiratory therapy device model; ii) a user interface type; iii) a user interface model; iv) a conduit type; v) a conduit model; or vi) any combination of i-v. In some cases, the method further comprises: receiving sensor data from one or more sensors, wherein the sensor data is associated with a user engaging in a sleep session, wherein outputting of the simulated respiratory therapy device sound occurs during the sleep session; determining sleep performance information using the sensor data; and outputting the sleep performance information. In some cases, the method further comprises modifying output of the simulated respiratory therapy device sound during the sleep session. In some cases, modifying output of the simulated respiratory therapy device sound is based on the determined sleep performance information. In some cases, generating the simulated respiratory therapy device sound is associated with a first respiratory therapy device model, the method further comprising: retrieving historical sleep performance information associated with a historical sleep session, wherein the historical sleep session occurred during outputting of additional simulated respiratory device sound, wherein the additional simulated respiratory device sound is associated with a second respiratory therapy device model; and generating a comparison between the sleep performance information and the historical sleep performance information. In some cases, the method further comprises generating a recommendation for the first respiratory therapy device model or the second respiratory therapy device model based on the generated comparison. In some cases, the method further comprises: receiving medical information associated with an individual; and modifying output of the simulated respiratory therapy device sound based on the received medical information.

Certain aspects and features of the present disclosure relate to a system comprising: a control system including one or more processors; and a memory having stored thereon machine readable instructions; wherein the control system is coupled to the memory, and any one of the methods described above is implemented when the machine executable instructions in the memory are executed by at least one of the one or more processors of the control system.

Certain aspects and features of the present disclosure relate to a system for shared sleep scoring, the system including a control system configured to implement any one of the methods described above.

Certain aspects and features of the present disclosure relate to a system for controlling respiratory therapy, the system including a control system configured to implement any one of the methods described above.

Certain aspects and features of the present disclosure relate to a system for simulating respiratory therapy, the system including a control system configured to implement any one of the methods described above.

Certain aspects and features of the present disclosure relate to a computer program product comprising instructions which, when executed by a computer, cause the computer to carry out any one of the methods described above. In some cases, the computer program product is a non-transitory computer readable medium.

### BRIEF DESCRIPTION OF THE DRAWINGS

The specification makes reference to the following appended figures, in which use of like reference numerals in different figures is intended to illustrate like or analogous components.

FIG. 1 is a functional block diagram of a system suitable for scoring sleep performance, according to certain aspects of the present disclosure.

FIG. 2 is a perspective view of the system of FIG. 1, a user, and a bed partner, according to certain aspects of the present disclosure.

FIG. 3 illustrates an example timeline for a sleep session, according to certain aspects of the present disclosure.

FIG. 4 illustrates an example hypnogram associated with the sleep session of FIG. 3, according to certain aspects of the present disclosure.

FIG. 5 is a perspective view of a pair of cohort members, including a first cohort member and a second cohort member, according to certain aspects of the present disclosure.

FIG. 6 is a flowchart depicting a process for generating and presenting sleep performance metrics for a sleep cohort according to certain aspects of the present disclosure.

FIG. 7 is a flowchart depicting a process for tracking goals for a sleep cohort according to certain aspects of the present disclosure.

FIG. 8 is a flowchart depicting a process for generating coaching suggestions for a sleep cohort according to certain aspects of the present disclosure.

FIG. 9 is a flowchart depicting a process for generating a simulated respiratory therapy device sound according to certain aspects of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Certain aspects and features of the present disclosure relate to evaluating the sleep performance of a cohort of multiple individuals sleeping in a shared environment (e.g., a bed, a room, a set of adjacent rooms, or a household). Individual or concerted sleep performance scores can be determined, as well as other sleep performance metrics. The evaluation of sleep performance for the entire cohort can be useful when at least one of the individuals is being treated (e.g., with a respiratory therapy system) for a sleep-related and/or respiratory disorder. Evaluation of the cohort can help identify actions that can be taken to improve the sleep performance of all individuals in the cohort.

Certain aspects and features of the present disclosure also relate to using the sleep data for one individual in an environment to adjust parameters of a respiratory therapy device used by another individual (e.g., a therapy user) in the same environment. In some cases, these adjustments, which can be automatically or manually implemented, can improve sleep quality for one or all individuals, as well as improve compliance and engagement with the respiratory therapy system by the user being treated by the system.

Certain aspects and features of the present disclosure also relate to generating simulated respiratory therapy device sounds to help an individual become accustomed to sleeping in an environment in which a respiratory therapy device is being used. Such a simulation can be helpful to acclimate a first individual to respiratory therapy system sounds when a second individual who sleeps in a shared environment with the first individual is using or will be starting respiratory therapy treatment. Similarly, the simulation can be helpful to acclimate the second individual, that is, a prospective user of the respiratory therapy system, in advance of commencing respiratory therapy with a respiratory therapy system. In some cases, such simulations can be tailored to the settings that are or will be used with the respiratory therapy system. In some cases, such simulations can be tailored to sleep performance of an individual in the environment. The sleep performance of an individual in the environment can be tracked to determine the effect the simulated respiratory therapy system sounds have on the individual's sleep performance.

Certain aspects of the present disclosure are useful to determine sleep performance metrics (e.g., a concerted sleep performance score and/or individual sleep performance scores) associated with a sleep session of a user receiving respiratory therapy (e.g., a therapy user) and a sleep session of a different individual sleeping in the same environment. This other individual sleeping in the shared environment with the therapy user can be known as a therapy-adjacent individual, a bed partner (e.g., in cases where the individual shares the same bed as the therapy user), or simply an individual. The therapy-adjacent individual can themselves be undergoing sleep-related therapy, such as therapy with a respiratory therapy system, although that need not always be the case. The therapy-adjacent individual can be physically adjacent the therapy user, although that need not always be the case. In some cases, the therapy-adjacent individual can be spaced apart from the therapy user, and in some cases even in a different room than the therapy user. As used with respect to a therapy-adjacent individual, the term “therapy-adjacent” is inclusive of an individual who sleeps in the same environment as the therapy user, and whose sleep performance may affect or be affected by the therapy user.

The therapy user and therapy-adjacent individual may sleep in a shared environment. The shared environment can be any environment within which the actions of the therapy user or the therapy-adjacent individual would affect the sleep performance of the other. In an example, the environment can be a bed, such as when the therapy user and therapy-adjacent individual are bed partners. In another example, the environment can be a room, such as when the therapy user and therapy-adjacent individual sleep in the same room, but in different beds. In another example, the environment can be adjacent rooms, such as when the therapy user and therapy-adjacent individual sleep in neighboring rooms, such as rooms that share a common wall. In another example, the environment can be a household, such as when the therapy user and the therapy-adjacent individual sleep in spaced apart rooms in a single structure. Other environments can be used.

In an example, a therapy user and therapy-adjacent individual may be spouses, in which case the sleep-related actions of one spouse may affect the sleep performance of the other (e.g., one spouse coming to bed later than the other spouse may awaken the other spouse from sleep). In another example, a therapy user may be a parent and the therapy-adjacent individual may be a child who sleeps in an adjacent room, in which case the sleep-related actions of the parent or child may affect the sleep performance of the other (e.g., loud snoring of the parent may affect the child's ability to fall asleep). In some cases, a concerted sleep performance score can be determined for a therapy user and multiple therapy-adjacent individuals (e.g., a concerted sleep performance score for an individual, the individual's spouse, and the individual's child or children). As used herein, reference to a single therapy-adjacent individual can include reference to multiple therapy-adjacent individuals, as appropriate. For example, where certain aspects and features of the disclosure describe sleep performance data of a therapy-adjacent individual as being used to adjust parameters of the therapy user's therapy device, in some cases sleep performance data of multiple therapy-adjacent individuals can be used to adjust parameters of the therapy user's therapy device.

As used herein, the group consisting of the therapy user and the therapy-adjacent individual can be known as a sleep cohort. The sleep cohort can include any number of members, including the therapy use and any number of therapy-adjacent individuals. Therefore, certain aspects and features of the present disclosure relate to calculation of sleep performance metrics (e.g., a concerted sleep performance score) for a sleep cohort that includes a therapy user and one or more therapy-adjacent individuals.

A variety of sleep performance metrics can be calculated, such as includes i) a concerted sleep performance score; ii) an individual sleep performance score associated with the therapy-adjacent individual; iii) an individual sleep performance score associated with the therapy user; iv) a hypnogram associated with the therapy-adjacent individual; v) a hypnogram associated with the therapy user; vi) a therapy score for the therapy user; vii) a resonance score (e.g., a score indicative of alignment between the sleep sessions of the therapy user and a therapy-adjacent individual); or viii) any combination of i-vii. Other sleep performance metrics can be used. Sleep performance metrics can be based on one or more aspects of an individual's sleep such as one or more of total sleep time, sleep efficiency, number of arousals and/or awakenings, total time in REM sleep, total time in deep sleep, sleep latency, REM latency, and wake after sleep onset (WASO).

A resonance score can be indicative alignment between the sleep sessions of two members or more of a sleeping cohort, as measured by any suitable metric or combination of metrics. In some cases, alignment can be based on the proximity in time of sleep onset times of the members. In some cases, alignment can be based on similarity between sleep performance metrics, such as sleep performance scores and/or sleep quality scores. Other metrics can be used to establish a resonance score.

A concerted sleep performance score is an indication of the overall sleep performance for the sleep cohort. Generally, as all members of the sleep cohort achieve improved sleep, the concerted sleep performance score will increase. In some cases, the concerted sleep performance score can be calculated based on individual sleep performance scores for the therapy user and the therapy-adjacent individual, although that need not always be the case. In some cases, the concerted sleep performance score can be an average or sum of the individual sleep performance scores of the therapy user and therapy-adjacent individual, although other formulae and measures can be used. Individual sleep performance scores can be determined based on sensor data, such as sensor data that is specific to the therapy user or specific to the therapy-adjacent individual. In some cases, however, a concerted sleep performance score can be based directly from the sensor data, without necessarily including determination of an individual sleep performance score.

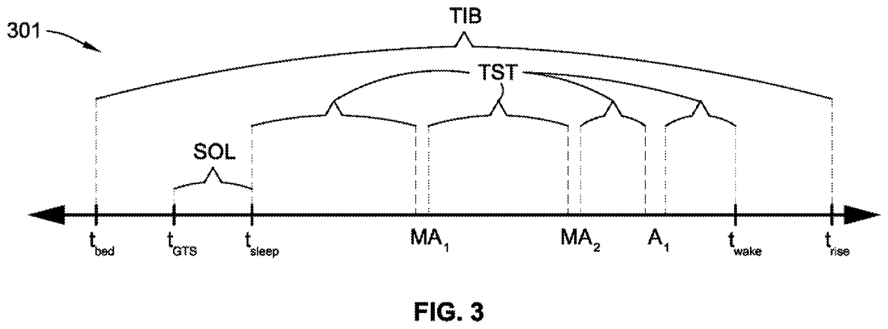

Sleep performance scores, including individual sleep performance scores and concerted sleep performance scores, can be based on sleep sessions of members of the sleep cohort. Each member of the sleep cohort can undergo an individual sleep session, as described in further detail herein with reference to FIG. 3, which may or may not overlap with the individual sleep session(s) of other member(s). In some cases, a cohort sleep session can be defined as a sleep session extending from the earliest initial start time of all the cohort members' sleep sessions to the latest end time of all the cohort members' sleep sessions. Therefore, within an overall cohort sleep session, a therapy user can undergo a therapy-user sleep session and a therapy-adjacent individual can undergo a therapy-adjacent individual sleep session. The therapy-user sleep session and therapy-adjacent individual sleep session can be entirely separate, can overlap, or can be identical. Thus, a concerted sleep performance score for a sleep cohort can be a score associated with a cohort sleep session.

Sensor data can include data from one or more sensors, such as sensors in or around a cohort member, in or around the entire cohort of sleepers, or in or around the environment. When processed, different sleep performance data can be extracted from the sensor data, which can be used to determine various sleep performance metrics. For the purposes of determining sleep performance metrics, the sensor data can be processed to obtain sleep performance data such as therapy information and sleep quality information. Therapy information can include information associated with the therapy user's use of a therapy system, such as a respiratory therapy system. For example, therapy information can include usage information (e.g., data indicative of the therapy user's use of the therapy device) and event information (e.g., data indicative of a sleep apnea or other event such as user interface on/off events and leak events). Sleep quality information can include objective data and/or subjective data about a cohort member's quality of sleep. Objective data can include data such as sleep state information (e.g., data indicative of whether or not the user is asleep) and/or sleep stage information (e.g., data indicative of the user's stage of sleep). Subjective data can include subjective feedback provided by the given cohort member or another member of the cohort. For example, data provided by the therapy-adjacent individual can be a response to the inquiry “On a scale of 1 to 5, how well rested do you feel?” upon waking; and data provided by the therapy user can be a response to the inquiry “On a scale of 1 to 5, how well do you believe your bed partner slept last night?” Sensor data can thus include objective measurements obtained from sensors such as radiofrequency sensors, microphones, pressure monitors, and the like; as well as data (e.g., feedback responses) obtained via sensors such as touchscreens, interactive buttons, and the like.

Therapy information can be data associated with the therapy user's use of a sleep-related therapy device. Any suitable sleep-related therapy device can be used. In some cases, the sleep-related therapy device can include adjustable parameters, such as dynamically adjustable parameters and/or manually adjustable parameters. In some cases, a sleep-related therapy device can include a respiratory therapy device, a mandibular repositioning device, a sleep-therapy implant (e.g., an implantable stimulator for stimulating the hypoglossal nerve in the neck), or other such device. Certain aspects and features of the present disclosure can be especially useful when the therapy device used by the therapy user is a respiratory therapy device.

A respiratory therapy system can include a respiratory therapy device that supplies pressurized air to the therapy user via a conduit and user interface. Different models and types of conduits, as well as different models and types of user interfaces, can be used to fluidically couple the therapy user to the respiratory therapy device. While receiving the respiratory therapy, the therapy user can engage in a sleep session, during which first sensor data can be collected from a first set of one or more sensors, such as sensors in the respiratory therapy device, sensors in a user device (e.g., smartphone), sensors in an activity tracker (e.g., wearable activity tracker), or other sensors located in, on, or around the therapy user (e.g., implantable devices, clothing-integrated sensors, mattress-integrated sensors, wall-mounted or ceiling-mounted sensors, or the like). The first sensor data collected from the first set of one or more sensors can be used to determine therapy information (e.g., one or more usage variables associated with use of the respiratory therapy system) associated with the therapy user. The first sensor data can also be used to determine sleep quality information (e.g., sleep state information, sleep stage information, and/or subjective feedback) associated with the therapy user. Other variables and/or information can be determined using the first sensor data.

For a therapy-adjacent individual, second sensor data can be collected from a second set of one or more sensors. The second set of one or more sensors can be the same as the first set of one or more sensors, can be a subset of the first set of one or more sensors, can be a superset of the first set of one or more sensors, can overlap with the first set of one or more sensors (e.g., share some sensors, but not all sensors, with the first set of one or more sensors), or can be an exclusive set of one or more sensors (e.g., share no sensors with the first set of one or more sensors). Likewise, the second sensor data can be the same as the first sensor data, can be a subset of the first sensor data, can be a superset of the first sensor data, can share some sensor data with the first sensor data, or can be share no sensor data with the first sensor data. The second sensor data can be collected simultaneously with or in temporal proximity to the first sensor data. In an example with an exclusive set of one or more sensors, sleep performance for a therapy user can be evaluated using sensor data from the therapy user's respiratory therapy device, and optionally from the therapy user's smartphone, while sleep performance for the therapy-adjacent individual can be evaluated using sensor data from the therapy-adjacent individual's smartphone. Other devices and sensors can be used.

Therapy information can include usage variables associated with use of the respiratory therapy system. Usage variables associated with use of the respiratory therapy system can include any suitable variable related to how a therapy user makes use of the respiratory therapy system. Examples of suitable usage variables include usage time (e.g., a duration of time the therapy user makes use of the respiratory therapy system); a seal quality variable (e.g., an indication of the quality of seal between the therapy user and the user interface); a leak flow rate variable (e.g., an indication of the rate of flow of unintentional leaks, such as leaks through a poor-quality seal or mouth-breathing while wearing a nasal pillow type user interface); event information (e.g., an indication of detected events that occurred during the sleep session, such as an apnea-hypopnea index (AHI)); user interface compliance information (e.g., an indication of detected user interface transition events, such as donning or removing the user interface); a number of therapy sub-sessions within the sleep sessions (e.g., a number of separate blocks of continuous usage of the respiratory therapy system); and user interface pressure. Other usage variables can be used. Statistical summaries (e.g., averages, maximums, minimums, counts, and the like) of one or more usage variables can be used as one or more additional usage variables. The one or more usage variables can include any suitable combination of usage variables.

Determining a usage variable can include processing sensor data to identify one or more values associated with the usage variable. The one or more values can be a measurement or calculated score associated with the usage variable. For example, a seal quality variable can be a measurement of leak flow rate (e.g., in L/min) or a seal quality score (e.g., 18 out of 20). Determining a usage variable can include determining a single value or multiple values (e.g., timestamped values). For example, in some cases, determining a seal quality variable can include determining a single value representative of the overall (e.g., average) seal quality throughout the sleep session (e.g., 18 out of 20). In some cases, however, determining a seal quality variable can include determining a set of timestamped values representative of the seal quality over time (e.g., on a scale of 0 to 20, 18 at 10:00:00 PM, 18.1 at 10:00:05 PM, 18.2 at 10:00:10 PM, and the like), such as data that can be charted to depict seal quality throughout a duration of time.

Sleep quality information can include objective information, such as sleep state information, sleep stage information, and/or other such information obtained from the sensor data; as well as subjective information, such as subjective feedback received in response to the presentation of feedback questions to a user. Sleep state information is information indicative of the sleep state of the cohort member. The sleep state is indicative of whether or not the cohort member is asleep. Sleep stage information can include information indicative of the sleep stages undergone by the cohort member during the sleep session. Examples of sleep stages include a wakefulness stage, a rapid eye movement (REM) stage, a light sleep stage, and a deep sleep stage. Sensor data can be processed to determine times when the cohort member enters and exits various stages of sleep. In some cases, determining sleep stage information can include determining a total duration of time the cohort member spent in each sleep stage. In an example 8-hour sleep session, the sleep stage information may indicate a total of 21 minutes in wakefulness, 101 minutes in REM sleep, 267 minutes in light sleep, and 91 minutes in deep sleep. In some cases, however, determining sleep state information and/or sleep stage information can include generating timestamped data indicative of the sleep state and/or sleep stage of the cohort member at various times throughout the sleep session. Timestamped sleep stage information can be charted to generate a hypnogram of the cohort member's sleep session.

In some cases, the objective data can include various parameters extracted from the sensor data, such as a total time in bed, a total sleep time, a sleep onset latency, a wake-after-sleep-onset parameter, a sleep efficiency, number of arousals and/or awakenings, a fragmentation index, total time in REM sleep, REM latency, total time in deep sleep, or any combination thereof.

For therapy-adjacent individuals, a sleep performance metrics may be based solely on sensor data that is sleep quality data (e.g., data that is related to the therapy-adjacent individual's sleep session, but not specifically related to the use of a therapy device), which can include subjective data in the form of subjective feedback. In such cases, sleep performance metrics can include or be based on various factors associated with the therapy-adjacent individual's sleep, such as sleep stage information, including time spent in various sleep stages, as well as subjective feedback, such as indications of how well-rested the user feels at or after the conclusion of the sleep session.

In cases where subjective feedback is used, subjective feedback for a particular cohort member (e.g., therapy user) can include subjective feedback obtained from that particular cohort member (e.g., feedback provided by the therapy user) and/or subjective feedback obtained from another member of the cohort (e.g., feedback provided by the therapy-adjacent individual).

Sleep quality data can be used to determine a sleep quality score or other sleep quality metrics. The sleep quality score can be an indication of the quality of sleep undergone by the cohort member during the sleep session. For example, a sleep session with many awakenings or interruptions may have a low sleep quality score, whereas a sleep session with fewer awakenings or interruptions may have a higher sleep quality score. In some cases, the sleep quality score can be based on subjective feedback (e.g., feedback from a cohort member indicating a subjective feeling of restfulness following a sleep session), can be based on objective data, or a combination of the two. As used herein, subjective feedback can also encompass daytime information such as subjective energy levels, fatigue levels, mood (e.g., content, irritable, etc.), etc. Some such information can also be collected objectively via sensors, such as wearable sensors that detect cardiac, respiratory and/or movement parameters from which energy levels, fatigue levels, mood, etc. can be inferred.

In an example, sleep quality score or a component thereof can be determined objectively, such as based on sleep stage information. In an example, time spent in different sleep stages can be used to determine a sleep quality score. Additionally or alternatively, the pattern of sleep stages (e.g., the sleep architecture) can be used to determine a sleep quality score. The sleep stage information can be segmented into sleep stage segments indicative of time spent in each sleep stage (e.g., a total time spent in each sleep stage during a sleep session or durations for each of the consecutive sleep stages that occur in the sleep session).

In some cases, sleep quality score can be based at least in part on physiological data associated with the cohort member, such as i) respiration rate; ii) heart rate; iii) heart rate variability; iv) movement data; v) electroencephalograph data; vi) blood oxygen saturation data; vii) respiration rate variability; viii) respiration depth; ix) tidal volume data; x) inspiration amplitude data; xi) expiration amplitude data; xii) inspiration volume data; xiii) expiration volume data; xiv) inspiration-expiration ratio data; xv) perspiration data; xvi) temperature data; xvii) pulse transit time data; xviii) blood pressure data; xix) position data; xx) posture data; xxi) blood sugar level data; or xxii) any combination of i-xxi.

For the therapy user or a therapy-adjacent individual that happens to also make use of a sleep-related therapy device, sleep performance metrics may be based on sensor data that includes therapy information and/or sleep quality information. In some cases, therapy information and sleep quality information can be used in combination to determine sleep performance metrics. For example, it can be informative and useful to track a total amount of time that the therapy user makes use of a respiratory therapy device during a sleep session (generally, the more time used, the better) alongside sleep stage information. In another example, since apnea and hypopnea events may be more prevalent (e.g., because of decreased tone of the genioglossus muscle in the tongue) during REM sleep and more detrimental (e.g., due to the chance of interrupting REM sleep, negatively impacting spatial memory, and/or reducing amount of deep sleep) during REM and deep sleep, it may be more useful to track an amount of time the respiratory therapy device is used during REM sleep and/or during deep sleep. Thus, in addition to tracking overall usage time, the amount of time the respiratory therapy device is used in certain sleep stages (e.g., REM sleep or deep sleep) can be emphasized (e.g., weighted more strongly) than time the respiratory therapy device is used in other sleep stages (e.g., awake or light sleep). Thus, even if a therapy user makes use of a respiratory therapy device for longer periods of time before falling asleep, the sleep performance score may not increase much or at all. However, if the same therapy user makes use of the respiratory therapy device for longer periods of REM stage sleep, the sleep performance score may increase substantially.

The therapy information can be used to determine a therapy score. The therapy score can be indicative of the therapy user's use of the therapy, such as the effectiveness of the therapy and/or the therapy user's adherence to the therapy.

Sleep quality scores and therapy scores can be used as components for sleep performance scores. In some cases, sleep quality scores and therapy scores can be further broken down into subcomponents, such as a score for time spent in REM sleep, a score for length of a sleeping session, a score for time spent using the therapy device, and a score for the presence or absence of unintentional leaks, and the like. Thus, a sleep quality score and/or a therapy score, and optionally one or more subcomponent scores, can be used as sleep performance metrics or can be used to generate sleep performance metrics. In some cases, the various component scores, and optionally subcomponent scores, for members of a cohort can be used to generate individual sleep performance scores, which can be presented as individual sleep performance scores and/or optionally used to generate a concerted sleep performance score. In some cases, the various component scores, and optionally subcomponent scores, for members of a cohort can be used to directly generate a concerted sleep performance score, without necessarily first generating individual sleep performance scores.

In an example, a sleep performance score can be presented using a numerical score, although that need not always be the case. In some cases, a concerted sleep performance score can be presented using a graphical device indicating equilibrium between two or more components or subcomponents. For example, a concerted sleep performance score can be presented as an equilibrium between the individual sleep performance scores of two members of a sleep cohort. In an example, the graphical device can be a bubble level or similar device, allowing an individual to quickly and easily see which of the components/subcomponents/members is performing relatively better than the other, and optionally how significantly better. If one individual achieves a much higher sleep performance score than the other, the graphical device may show strong disequilibrium, whereas a slightly higher score would only show a slight or no disequilibrium. In some cases, multiple presentation techniques can be combined (e.g., presenting the sleep performance score as a numerical score alongside a graphical device indicating equilibrium).

Calculation of a concerted sleep performance score (e.g., a combined sleep performance score) can include the calculation of individual scores for the therapy user and the therapy-adjacent individual; or can include the calculation of a single, concerted sleep performance score using the various sensor data and/or subjective feedback received from the therapy user and the therapy-adjacent individual. Sleep performance scores and other sleep performance metrics can be provided in any suitable fashion, such as numbers on a scale (e.g., a number on a scale of 0 to 100), data on a chart (e.g., a hypnogram of sleep stage data), information represented by a graphic (e.g., a green check mark indicating no detected events), or otherwise.

In an example, a concerted sleep performance score can be presented as a number on a scale of 0 to 100, with higher numbers indicated higher quality sleep for the sleep cohort (e.g., higher quality sleep for the therapy user and any number of therapy-adjacent individuals, as a whole). In some cases, a concerted sleep performance score can be an average of individual sleep performance scores. In an example, a therapy user can have a sleep performance score of 70 and a therapy-adjacent individual can have a sleep performances score of 80, in which case the concerted sleep performance score may be 75. In that same example, if the therapy user achieves a subsequent sleep performances score of 78 and the therapy-adjacent individual achieves a subsequent sleep performances score of 76, the concerted sleep performance score may be 76. Thus, it may be possible for one cohort member's sleep performance score to drop while the concerted sleep performance score increases. In some cases, concerted versions of other sleep performance metrics can be calculated, such as in a fashion similar to how the concerted sleep performance score is calculated form individual sleep performance scores. As described herein, a concerted sleep performance score can also or additionally be presented in other fashions, such as using a graphical device indicative of equilibrium between the therapy user's sleep performance score and the therapy-adjacent individual's sleep performance score.

Sleep performance metrics can be presented to a cohort member in any suitable fashion, such as via a display device on a respiratory therapy device, a display device on a user device (e.g., a smartphone), or otherwise. Presentation of any sleep performance metric can include presenting the sleep performance metric and underlying component and/or subcomponent scores. For example, presenting a sleep performance metric can include presenting i) a concerted sleep performance score; ii) an individual sleep performance score for the given cohort member; iii) an individual sleep performance score for another member of the cohort; iv) one or more component and/or subcomponent scores that make up any of the sleep performance score of i-iii; or v) any combination of i-iv. Examples of component scores and/or subcomponent scores include scores for each of the usage variable(s), the sleep stage information, the sleep state information, and/or the sleep quality information. In some cases, presenting a sleep performance metric can include presenting a graphical representation of the components and/or subcomponent s scores that make up sleep performance metric. For example, presentation of a concerted sleep performance score can include presenting a graphical representation of the individual sleep performance scores that make up the concerted sleep performance score.

In some cases, presenting a sleep performance metric can include presenting an amount of contribution a particular component or subcomponent made to the overall sleep performance metric. In some cases, components or subcomponents, such as usage variables, can be broken down (e.g., binned) and/or sorted by sleep stage information. For example, a set of four subcomponent scores (e.g., bins) may be presented for a usage time variable, including a score for usage time during wakefulness, a score for usage time during REM sleep, a score for usage time during light sleep, and a score for usage time during deep sleep. It should be understood that each of the subcomponent scores can be a score that is calculated by applying a weighting value to the usage variable as described herein with reference to calculating an overall sleep performance score.

Sleep performance metrics, such as a sleep performance score, can act as an objective measurement of the cohort member's sleep session. In some cases, for a therapy user, the sleep performance metrics can be limited to only that portion of the therapy user's sleep session during which respiratory therapy was used, although that need not always be the case. Sleep performance metrics can provide information to a therapy user to help monitor, maintain, and/or encourage self-compliance (e.g., use of the respiratory therapy device as desired or prescribed) and/or can provide information to a therapy-adjacent individual to help monitor, maintain, and/or encourage the therapy user's compliance. In some cases, sleep performances metrics can provide information to healthcare providers, facilities, and/or healthcare-related companies (e.g., healthcare insurance providers) about the compliance and efficacy of a therapy user making use of the respiratory therapy device during sleep, and/or the effect the therapy user or therapy-adjacent individual has on the other's sleep. In some cases, sleep performance metrics can be used to provide objective measurements for research purposes and evaluation.

In some cases, goals can be established to improve overall sleep quality or improve certain aspects related to sleep quality (e.g., to improve certain specific sleep performance metrics). In some cases, a cohort member can establish a goal directly, such as via a graphical user interface on an app running on a user device (e.g., smartphone). In some cases, the cohort member can select from a list of suggested goals, such as a list of global preset goals (e.g., goals commonly selected by all users), a list of demographic-specific preset goals (e.g., goals commonly selected by users who share certain demographics with the cohort member), or a list of custom-generated goals (e.g., goals custom-generated for the cohort member). Custom-generated goals can be generated automatically based on sensor data (e.g., sensor data from historical sleep sessions) or based on member-provided input. When provided with a list of suggested goals, a cohort member can select one or more goals to use.

In an example where custom-generated goals are automatically based on sensor data, historical sleep performance metrics (e.g., historical concerted sleep performance scores) can be analyzed to determine one or more factors that are likely to be affecting a particular sleep performance metric. This analysis can include using historical sensor data to identify the factor(s) in question, then identify a suggested action to take to improve a future sleep performance metric (e.g., a future concerted sleep performance score). A suggested action can include the performance of a given action (e.g., brush teeth before going to sleep) or the avoidance of performing a given action (e.g., avoid ingesting caffeine two hours before going to sleep). The factors in question can include factors that may influence the given sleep performance metric. Examples of factors can include loud snoring, an obstructive sleep apnea diagnosis, AHI, cohort members having disparate work shifts, cohort members including young children, poor sleep hygiene, a cohort member's anxiety about their own sleep or the sleep of another cohort member, consumption of caffeine, consumption of alcohol, and others. In some cases, the one or more factors can be determined from feedback supplied in response to a questionnaire, such as a questionnaire asking about potential pain points associated with a member of the cohort being a therapy user. Example pain points include worry that a bed partner would have to sleep in another room, worries about health, worries about determining if therapy is sufficient, worries about not getting sufficient therapy (e.g., if the user interface is removed during a sleep session), worries about therapy device settings (e.g., pressure levels, noise, leaks, and the like), worries about having to contact medical equipment suppliers and manufacturers. In some cases, the questionnaire can be based on a clinically validated questionnaire (E.g., the Epworth Sleepiness Scale, the quality of life index, the Dyadic adjustment scale, or the Beck depression inventory).

The suggested actions can be related to the factors. For example, for a poor sleep hygiene factor, the suggested action may be to stop using electronic screens at least 30 minutes prior to going to sleep, not eating at least 60 minutes prior to going to sleep, and other such actions. As another example, for a factor where the cohort member experiences anxiety about the sleep of another cohort member, the suggested action may be to go through an anxiety-reducing exercise, to discuss the anxiety with the other cohort member, or the like. The list of suggested goals can be goals related to taking the suggested action. For example, if analysis of historical concerted sleep performance scores identifies that when the therapy user falls asleep more than 30 minutes prior to the therapy-adjacent individual goes to bed, the concerted sleep performance score tends to be lower, then a suggested action may be to have the therapy-adjacent individual go to bed within 30 minutes of the therapy user falling asleep, and the goal can be for the therapy-adjacent individual to go to bed within 30 minutes of the therapy user falling asleep for at least 75% of the sleep sessions for the next two weeks.

In another example where custom-generated goals are automatically based on sensor data, historical therapy information can be analyzed to identify historical respiratory therapy device usage information. The historical respiratory therapy device usage information can be further analyzed to identify one or more goals. For example, if analysis of the historical respiratory therapy device usage information identifies that concerted sleep performance scores improve when the respiratory therapy device is used for at least five hours during a sleep session, the suggested action may be to use the respiratory therapy device for at least five hours each sleep session, and the list of suggested goals may include a first goal to use the respiratory therapy device for at least five hours each sleep session for the next five days; a second goal to use the respiratory therapy device for at least eight hours for three sleep sessions over the next week; and a third goal to use the respiratory therapy device for at least five hours for seven consecutive days sometime within the next three months.

In some cases, the cohort member can be provided with a questionnaire containing one or more questions. The cohort member's responses to these questions can be used to generate a list of suggested goals and/or identify one or more particularly relevant goals. In some cases, the responses of some or all of the members of a cohort can be used to generate a list of suggested goals.

In some cases, a cohort member can provide input in the form of suggestive feedback associated with one or more historical sleep sessions. This subjective feedback can be used to identify one or more goals. For example, if feedback that the therapy-adjacent individual does not feel well rested coincides with sleep sessions in which the therapy user sleeps in different room than the therapy-adjacent individual, a goal may be suggested for the therapy user and therapy-adjacent individual to sleep in the same room for at least a threshold number of nights a week. This example goal can be evaluated by estimating a distance between the therapy user and therapy-adjacent individual based on sensor data (e.g., by analyzing patterns in audio data, by analyzing signal strength for wireless signals, or the like).

Goals can be set for individual cohort members or for the entire cohort. For example, a single cohort member may have an individual goal of stopping the viewing of electronic screens by 9:30 pm, in which case that cohort member's goal may be individually tracked, however the entire cohort may have a goal to achieve a concerted sleep performance score of at least 90 out of 100.

Once a goal is established, the goal can be monitored and evaluated after every sleep session of a member of the cohort for whom the goal is set and/or after every cohort sleep session. Sensor data can be used to monitor and evaluate the goal. The evaluation of the cohort member's progress or the cohort's progress with respect to any given goal can be presented to one or more members of the cohort, such as via one or more display devices. This display of progress can help motivate cohort members to improve their sleeping hygiene and improve their overall sleep quality.

In an example, if a goal for members of a cohort is to go to sleep at approximately the same time as one another, the start times of the cohort members' respective sleep sessions can be monitored to determine whether or not they are starting their sleep session within a threshold amount of time from one another.

In some cases, receiving goal information can include receiving a target completion date associated with the goal. A target completion date can be received in the form of a specific date or a number of days from the current date. The target completion date can be manually provided, such as via user input, or can be automatically determined. An automatically determined target completion date can then be automatically set for a given goal, or can be suggested to a cohort member. Automatic determination of a target completion date can be based on any suitable data, such as i) the first sleep performance data; ii) the second sleep performance data; iii) historical sleep performance data; or iv) any combination of i-iii. The target completion date can be determined to be a target completion date that is achievable to the given cohort member or cohort. The achievability of the target completion date can be based on historical sleep performance data, such as by identifying previous instances where the goal was met, identifying trends in sleep performance metrics, or other analysis. For example, for a goal to not drink caffeine within two hours of going to sleep for four consecutive days, analysis of historical sleep performance data (e.g., including historical subjective feedback about caffeine usage) might identify that in the past, the cohort member was able to previously achieve the goal of not drinking caffeine within two hours of going to sleep for four consecutive days after two weeks of attempting to do so. Thus, the target completion date may be set aggressively with respect to the previous attempt (e.g., slightly less than two weeks from the current date), set equal to the previous attempt (e.g., set at two weeks from the current date), or set reservedly with respect to the previous attempt (e.g., slightly more than two weeks from the current date).

In some cases, a target completion date can be updated based on an estimated duration of time until the goal is achieved. This updating of the target completion date can be made to avoid discouraging sentiment that a cohort member might feel if they have not made enough progress by an approaching target completion date. A determination of an estimated duration of time until the goal is achieved can be based on sleep performance data from the therapy user for the current sleep session (e.g., last night's sleep session), sleep performance data from the therapy-adjacent individual for the current sleep session, and/or historical sleep performance data from one or all users for historical sessions. For example, if the current and historical sleep performance data associated with the therapy user shows that the therapy user is progressively using the respiratory therapy device for more time each night, but still less than expected for a set goal's upcoming target completion date, an estimation can be made as to when the therapy user is likely to achieve the goal. Then, this estimation can be used to update the target completion date. The target completion date can be updated aggressively with respect to the estimation, equal to the estimation, or reservedly with respect to the estimation. In some cases, an estimation can be generated for other purposes, such as to help motivate cohort members to meet their goals on time or early, or to evaluate whether the cohort member is improving in their attempt to achieve the goal.

Examples of possible goals include using the therapy device for a certain number of hours per night, having a therapy user and therapy-adjacent individual sleep in the same environment at least a threshold number of nights per week, improving a sleep performance metric of the therapy user or a therapy-adjacent individual, improving a concerted sleep performance metric, improving subjective feedback (e.g., improving the response to a daily question of how well rested the cohort member feels), stopping or minimizing snoring, losing weight, improving mood, reducing sleepiness between sleep sessions, improving compliance of using the therapy device, improving compliance of using the system to monitor sleep performance (e.g., compliance of starting up a sleep monitoring app on a smartphone each night), and the like.

In some cases, an interactive feed can be provided to share sleep-related data (e.g., individual sleep performance scores) and/or goals between members of the cohort. The interactive feed can permit cohort members to comment on one another's entries, such as via text-based comments, image-based comments, or reactions. Reactions can include any number of preset responses (e.g., “likes,” “thumbs up,” various emojis, and the like). In some cases, reactions can be tallied, with a count of the number of reactions presented with the entry on the feed. In some cases, the interactive feed can provide further motivation to achieve a given goal. In some cases, sleep-related data and/or goals for a cohort member or other members of the cohort can be shared with outside individuals (e.g., individuals not within the sleep cohort, such as members of a different sleep cohort). Such sharing of data can be in a similar interactive feed, permitting individuals to comment on one another's entries and provide further motivation to achieve a given goal and obtain good sleep hygiene and good overall sleep quality.

In sharing data to an interactive feed, a cohort member's user device (e.g., smartphone) can transmit summary information based on sleep performance data. This summary information can include sleep-related data, member-provided comments, and the like. In some cases, this summary information can be transmitted directly to the user device of another cohort member. In other cases, this summary information can be transmitted to a network-accessible server (e.g., via a local area network, wide area network, cloud, or the Internet), which can then be accessed by the user device of another cohort member. When a server is used, unique identifiers (UIDs) associated with each of the members of the cohort can be correlated with one another directly or via a UID for the cohort.

In some cases, a coaching suggestion can be identified and provided to improve a sleep performance metric of a cohort member or a concerted sleep performance metric of a cohort. The coaching suggestion can be a recommendation to undertake a particular action or not undertake a particular action. In some cases, these actions can be similar to those associated with goal setting, as described herein, although that need not always be the case. The coaching suggestion can be generated automatically based on analysis of historical sensor data and/or historical sleep performance metrics; or can be generated manually, such as in response to subjective feedback.

When generated automatically, the coaching suggestion can be obtained by analyzing historical sleep performance data and historical sleep performance metrics to identify a factor that affects a given historical sleep performance metric, then identify a suggested action to take that is associated with the factor. The suggestion action can be selected as one that is expected to improve the given sleep performance metric in the future (e.g., improve a future sleep performance metric). The coaching suggestion can then be generated as a suggestion designed to have the cohort member take the suggested action. The suggested action can be the performance of a given action or the avoidance of performing a given action.

When generated manually, the coaching suggestion can be based on specific subjective feedback from a cohort member. For example, a cohort member can say that they want to go to sleep no later than 10 pm or can identify that they do not feel well-rested when falling asleep after 10 pm the night before. In such cases, coaching suggestions can be automatically generated to remind the cohort member to go to sleep by 10 pm or take other action to facilitate going to sleep by 10 pm.

In some cases, manually generated coaching suggestions can be provided for individual cohort members. For example, a coaching suggestion to remember to use a respiratory therapy device may be provided to only the therapy user.

Coaching suggestions can be direct or indirect. A direct coaching suggestion is indicative of the desired result. An indirect suggestion is not necessarily indicative of the desired result, but is expected to achieve the desired result. Indirect suggestions can be subliminal, implicit, or obfuscated. Subliminal suggestions are designed to achieve the desired result without the individual perceiving that the suggestion is intended to achieve the desired result. Implicit suggestions are designed to achieve the desired result by suggesting action that may be related to the desired result. The individual may perceive that the implicit suggestion is associated with the action related to the desired result, and the individual may perceive that implementing the action will improve the desired result, but the implicit suggestion does not directly indicate the desired result. In some cases, an indirect coaching suggestion can be in the form of a statement or questions, rather than explicitly stating an action to perform. For example, instead of explicitly suggesting that the therapy user use the respiratory therapy system on the next sleep session, an indirect coaching suggestion can be a reminder to check the fit of the user interface.

In an example where the desired result is to use the respiratory therapy system for more days out of the week, various coaching suggestions can be used. A direct coaching suggestion can be to use the respiratory therapy system for at least five sleep sessions this week. A subliminal coaching suggestion can be in the form of a series of prompts to the therapy user, such as providing motivating prompts each morning after the respiratory therapy system is used, thus subliminally motivating the therapy user to use the respiratory therapy system more often that week. An implicit suggestion can be presented as a notice showing how long the respiratory therapy system was used during the previous sleep session and suggesting that the therapy user attempt to meet or exceed that previous sleep session's use time. Thus, the implicit suggestion is directed towards improving length of time used, but also has the effect of increasing the chance the therapy user will use the respiratory therapy system during the next sleep session, and potentially subsequent sleep sessions.

Coaching suggestions based on subjective feedback or data associated with a particular cohort member can be obfuscated or non-obfuscated. In some cases, an obfuscated suggestion can be a form of indirect suggestion. In some cases, a non-obfuscated suggestion can be a form of direct suggestion. A non-obfuscated coaching suggestion is a suggestion that is indicative of the underlying subjective feedback or underlying data. For example, if subjective feedback is that the therapy-adjacent individual wants the therapy user to use the respiratory therapy device or the therapy-adjacent individual indicates that they do not feel well-rested on days following sleep sessions where the therapy user did not use the respiratory therapy device, a non-obfuscated coaching suggestion can be a reminder to the therapy user to user the respiratory therapy device to ensure the therapy-adjacent individual achieves high-quality sleep. Such a non-obfuscated coaching suggestion is indicative of the underlying sleep-related concern (e.g., the sleep quality of the therapy-adjacent individual). In the same situation, an obfuscated coaching suggestion may be a reminder to the therapy user to adjust straps of the user interface for a proper fit or a reminder to the therapy user that they used the respiratory therapy device for a certain number of minutes during the last sleep session, and may want to improve during the upcoming sleep session. Such obfuscated coaching reminders are not directly indicative of the underlying sleep-related concern, but can have an effect of improving the underlying sleep-related concern. Thus, while the suggestion to adjust straps of the user interface may not identify that the therapy-adjacent individual wants the therapy user to use the respiratory therapy device to achieve a higher quality of sleep, the suggestion still has the effect of bringing the respiratory therapy device front-of-mind to the therapy user, especially if provided near the start of a sleep session, which can result in the desired outcome of the therapy user making use of the respiratory therapy device.

In another example, a non-obfuscated coaching suggestion may be a suggestion for the therapy-adjacent individual to encourage the therapy user to use their treatment device for longer in the next sleep session because it was only used for one hour in the previous sleep session. Such a non-obfuscated coaching suggestion reveals data associated with the therapy user, namely the length of time the treatment device was used in the previous sleep session. In the same situation, an obfuscated coaching suggestion may be a suggestion for the therapy-adjacent individual to encourage the therapy user to use their treatment device for at least five hours during the next sleep session. This obfuscated coaching suggestion can provide a similar effect while not revealing the underlying data (e.g., the therapy user's data).

Coaching suggestions can be individual in nature, or can be associated with an entire cohort. For example, an individual coaching suggestion can be for a single cohort member to undertake or not undertake a particular action, whereas a cohort coaching suggestion can be for all members of the cohort to undertake or not undertake a particular action.

Coaching suggestions can be evaluated through subjective feedback or objective data. When subjective feedback is used, a cohort member can indicate that the suggestion they attempted was successful. For example, in response to a prompt asking if the member tried the suggestion and if they feel well rested, the member can respond with “yes, I tried the suggestion” and “yes, I feel well-rested.” When objective data is used, one or more sleep performance metrics from the previous sleep session can be analyzed and/or compared against historical sleep performance metrics to determine whether or not an improvement was made. The system can assume the suggestion was made or the system can prompt the member to indicate whether or not the suggestion was made. Thus, the effectiveness of the suggestion can be evaluated based on a detected change in a sleep performance metric.

In some cases, an incentive system can be used to provide additional incentive to individual members of a cohort or the entire cohort. Incentives can be based on meeting threshold sleep performance metrics (e.g., threshold sleep performance scores), achieving goals or goal milestones (e.g., quantitative progress towards a goal), and/or implementing coaching suggestions (e.g., confirming that a particular action is taken or not taken). In some cases, a cohort incentive can be provided based on the entire cohort's performance, such as upon achieving a threshold concerted sleep performance metric or achieving a cohort goal.

In some cases, incentives can be individually provided, such as upon an individual cohort member achieving a threshold individual sleep performance score or implementing an individual coaching suggestion. In some cases, a combination of individual incentives and cohort incentives can be provided. In an example, a therapy user may achieve their threshold individual sleep performance score, but a therapy-adjacent individual does not achieve their respective threshold individual sleep performance score and the cohort as a whole does not achieve the threshold concerted sleep performance score. In this example, only the therapy user receives the incentive, although that need not always be the case.

In some cases, an individual incentive may be obtained by a cohort member based on the sleep session of another cohort member. For example, a therapy user may only obtain a particular incentive if the therapy-adjacent individual achieves a sleep performance score above a threshold amount. In such cases, a cohort member may be incentivized to improve the sleep of others members of the cohort.

Incentives can be provided for individual sleep sessions or for longer durations of time (e.g., all sleep sessions within a week or all sleep sessions within a month). Within a longer duration of time, incentives can be based on achieving a desired outcome (e.g., achieving a threshold sleep performance score or achieving a goal) once, during every sleep session, or for at least a threshold number of sleep sessions.

Incentives can be provided by members of the cohort, by members of other cohorts, or by third parties. Any suitable incentives can be used. For example, incentives can be in the form of monetary awards, gift cards, gift items, discount codes to retailers, and the like. In some cases, the incentives can be sleep-related or otherwise related to a goal or coaching suggestion.

In some cases, incentives can dynamically change based on historical data (e.g., historical sleep performance scores) and/or current data. For example, if an incentive valued at $10 is offered to a cohort member, but it is determined through analysis of historical data that the cohort member is not improving their sleep performance score or moving away from a desired goal, the incentive can be automatically adjusted to increase (e.g., to $15) to provide a stronger incentive to improve sleep performance and overall quality of sleep.

In some cases, sensor data from a therapy-adjacent individual can be used to control parameters associated with the therapy user's therapy device (e.g., respiratory therapy device). This control can be dynamic (e.g., automatically during the same sleep session), automatic (e.g., automatically between the current sleep session and a subsequent sleep session), or manual (e.g., proposed parameter adjustments provided to the therapy user after the current sleep session). In an example of dynamic control, the air pressure supplied by the respiratory therapy device may be automatically adjusted to increase (e.g., to a more effective, but potentially louder, level) when the sensor data indicates that the therapy-adjacent individual is in a particular sleep state or sleep stage. In an example of manual control, if the sensor data shows that the therapy-adjacent individual has microawakenings whenever the therapy user's user interface seal quality drops below a threshold amount, a message may be presented to the therapy user to take action to improve the seal quality (e.g., by adjusting or replacing the conduit or user interface) before a subsequent sleep session.

In an example, a therapy user can begin using a respiratory therapy device before a therapy-adjacent individual starts a sleep session. Identifying that the therapy-adjacent individual is not yet attempting to go to sleep, the respiratory therapy device may operate using normal parameters. However, if the sensor data indicates that the therapy-adjacent individual is attempting to go to sleep, the respiratory therapy device may operate using adjusted parameters that result in the respiratory therapy device operating in a quieter fashion (e.g., with the motor of the flow generator operating at a lower speed). The respiratory therapy device can continue operating using the adjusted parameters until the sensor data indicates the therapy-adjacent individual has achieved a particular sleep state (e.g., asleep) or sleep stage (e.g., light sleep). In some cases, while the therapy-adjacent individual is still attempting to go to sleep, the respiratory therapy device can continue operating using the adjusted parameters for a preset duration before reverting to previous parameters or further parameters, such as in the event the therapy-adjacent individual does not fall asleep.

In some cases, sensor data associated with one cohort member can be collected from a different set of sensors from another cohort member. For example, sleep performance data for a therapy user might be obtained from one or more sensors incorporated into the therapy user's respiratory therapy device and user device, whereas sleep performance data for the therapy-adjacent individual might be obtained from one or more sensors incorporated into the therapy user's user device. In such cases, sensor data associated with the therapy user can be synchronized with sensor data associated with the therapy-adjacent individual. Synchronizing sensor data can include synchronizing data according to timestamps, according to commonly detected events (e.g., aligning data based on detection of a particularly loud snore), or any combination thereof. The resultant synchronized sensor data can be used to i) improve a signal-to-noise ratio of a particular piece of original sensor data (e.g., by identifying and filtering out noise or by identifying and amplifying a desired signal); ii) perform further analysis and/or obtain sleep performance metrics; iii) confirm a possible event detected using original sensor data; or iv) any combination of i-iii. In some cases, performing further analysis can include detecting a location of a cohort member with respect to the various sensors used to collect the original sensor data (e.g., sensors in the therapy user's smartphone and sensors in the therapy-adjacent individual's smartphone). Such location detection can be used to identify a location of a therapy user or therapy-adjacent individual in the environment (e.g., position in a bed or position in a room). In some cases, a parameter of the respiratory therapy device can be adjusted based on this location information.

Certain aspects and features of the present disclosure relate to the generation of simulated respiratory therapy device sounds. Such simulated sounds can be simulated by any suitable device, such as a user device (e.g., smartphone) that contains a speaker and a microphone. The speaker can be used to output the simulated respiratory therapy device sound, while the microphone can be used to monitor the outputted sound and make adjustments as necessary to ensure the simulation is accurate.

The simulated respiratory therapy device sound can be generated on demand (e.g., programmatically generated via electronic oscillators) or can be pre-recorded (e.g., a pre-recorded file containing electronically-generated sound or a pre-recorded file containing a recording of a physical respiratory therapy device). In some cases, the simulated respiratory therapy device sound to be output can be selected by an individual using the respiratory therapy device simulator (e.g., a user undergoing respiratory therapy, an individual planning to start respiratory therapy in the future, an individual who sleeps in the same environment as a user undergoing respiratory therapy, or an individual who sleeps in the same environment as an individual planning to start respiratory therapy in the future). Selection of a particular simulated respiratory therapy device sound can include selecting a model and/or type of respiratory therapy device, and optionally selecting one or more settings or parameters. For example, an individual who sleeps in the same environment as a future therapy user may be able to select the model and prescribed settings that the future therapy user will be using in the future. Based on the selection, the simulator can adjust the generated sound or select a particular pre-recorded file to accurately simulate the simulated respiratory therapy device sound associated with the individual's selection. In some cases, the simulated respiratory therapy device sound can be adjusted based on a therapy user's actual model and/or actual settings for their respiratory therapy device.

In some cases, an individual can adjust the output of the simulated respiratory therapy device sound (e.g., adjust the volume of the simulated respiratory therapy device sound). Based on this adjustment (e.g., the adjusted volume), a respiratory therapy recommendation can be provided. The respiratory therapy recommendation can include a respiratory therapy device model, a user interface type and/or model, a conduit type and/or model, a set of respiratory therapy device settings, or any combination thereof. For example, an individual can set the maximum volume level that individual would be willing to tolerate for comfortable sleep, then the simulator can provide a respiratory therapy recommendation for a particular user interface that would achieve the desired results.

In some cases, an individual can engage in a sleep session while the simulated respiratory therapy device sound is being output. In such cases, sensor data can be used to track the sleep performance of the individual while the individual experiences the simulated respiratory therapy device sound. Such sensor data can be used to determine sleep performance metrics, which can be used to identify how well the individual tolerates the simulated respiratory therapy device sound. This individual can be a future therapy user or a future therapy-adjacent individual. In some cases, sensor data can be used to automatically adjust the outputted simulated respiratory therapy device sound. For example, as the individual passes through different sleep stages, the simulated respiratory therapy device sound can be automatically adjusted to increase or decrease in volume or otherwise alter other characteristics of the sound (e.g., simulated unintentional leak sounds or other simulated sounds associated with use of a respiratory therapy device) based on the individual's current sleep stage. In some cases, the simulated respiratory therapy device sound can be adjusted to become more intrusive (e.g., have a louder volume or have other characteristics that may disrupt an individual's sleep) until a sleep performance metric of the individual falls below a threshold value. Thus, an individual's tolerance to different volumes and types of simulated respiratory therapy device sounds can be evaluated objectively using one or more sleep performance metrics.

In some cases, the simulated respiratory therapy device sound can be adjusted based on historical sleep performance information (e.g., historical sleep performance metrics and/or underlying sensor data) collected in a previous sleep session. In such cases, the simulated respiratory therapy device sound for the current sleep session can be different form the simulated respiratory therapy device sound used in the previous sleep session. Then, the historical sleep performance information can be compared with sleep performance information from the current sleep session to generate a comparison between the two different simulated respiratory therapy device sounds. This comparison can be presented to the individual. In some cases, a recommendation can be generated based on the comparison between the sleep performance information of the previous sleep session and the current sleep session. The difference in the simulated respiratory therapy device sounds can be due to the use of different respiratory therapy device models, difference in volume, or difference in other characteristics. Thus, the individual may be able to select a more desirable configuration based on sleep performance comparisons. For example, a future therapy user may try simulated respiratory therapy device sounds for a first respiratory therapy device on a first night, then try simulated respiratory therapy device sounds for a second respiratory therapy device on the second night. If the future therapy user's sleep performance metrics were improved on the second night, the future therapy user may opt to proceed with the second respiratory therapy device instead of the first respiratory therapy device.

In some cases, the simulated respiratory therapy device sound can be modified based on medical information associated with an individual. Such medical information can include, height, weight, gender, diagnoses, or other such information. For example, when initially starting up the simulator, the simulator can prompt the individual to answer certain questions (e.g., obstructive sleep apnea questions, such as the STOP-Bang questionnaire), then the simulator can use the answers to modify (e.g., alter and/or select) the simulated respiratory therapy device sound. For example, if the individual answers questions in a fashion that indicates a high likelihood of obstructive sleep apnea, the simulated respiratory therapy device sound may be modified to include characteristics associated with use of a respiratory therapy device by a person having obstructive sleep apnea.

Certain aspects and features of the present disclosure also relate to an interactive system for identifying pain points associated with a sleep cohort in which a member is a therapy user. Once identified, the system can provide information or coaching suggestions to ease the identified pain points. For example, if a cohort member answers a questionnaire in a fashion indicative of anxieties about the therapy and its influence on the cohort member's sleep quality, the system can provide information and/or coaching suggestions to assuage the cohort member's anxieties. For example, the system can provide knowledge and tips, interactive content to solve common doubts about the therapy (e.g., noise of the therapy device), conversation topics to help cohort members communicate concerns with one another, and/or cross-directed content associated with a questionnaire response by another member of the cohort. Such information and/or coaching suggestions can be in the form of text, sound, video, or any other form. In some cases, historical answers to questions can be used to generate new questions for future questionnaires. Questionnaires can be used as part of the subjective feedback for the sleep quality score, or can be used in other ways.

In some cases, the system can allow users to respond with free text or audio (e.g., via a microphone) that can be interpreted by a natural language processor. This interpretation can result in the extraction of useful information that can be stored in a structured format, optionally along with sentimental analysis of the input. In some cases, the system can allow users to respond with fixed choices (e.g., multiple choices, Likert scales, or graphical choices).

In some cases, the system can provide coaching suggestions related to the concerns of another member of the cohort.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

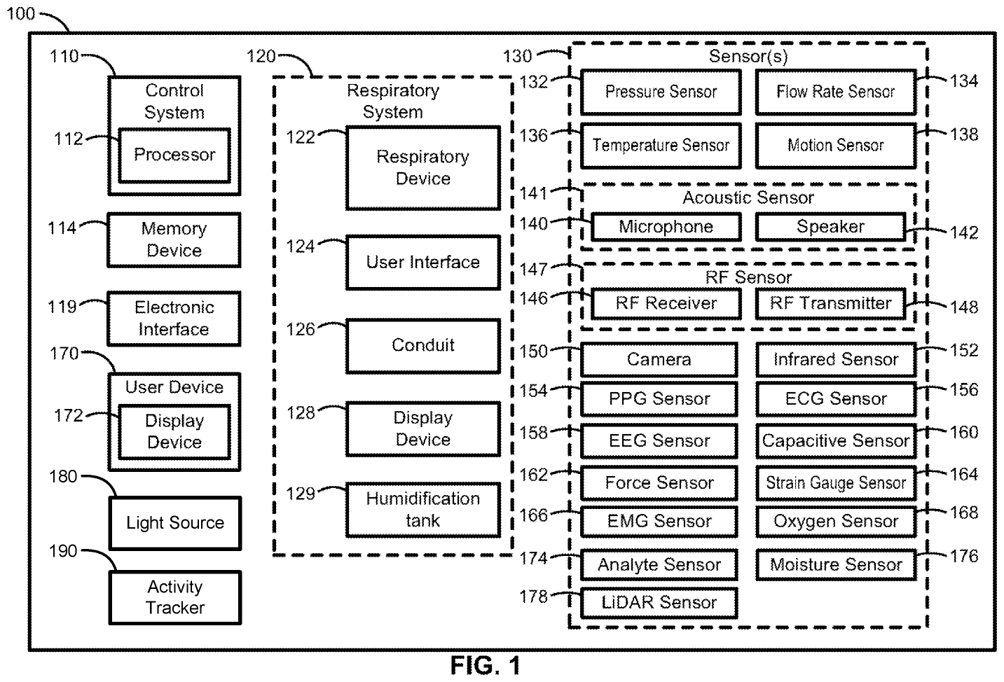

Referring to FIG. 1, a system 100, according to some implementations of the present disclosure, is illustrated. The system 100 includes a control system 110, a memory device 114, an electronic interface 119, a respiratory therapy system 120, one or more sensors 130, one or more user devices 170, one or more light sources 180, and one or more activity trackers 190.

In some cases, a single system 100 can be used to monitor multiple members of a sleep cohort. In some such cases, the single system 100 can include multiple user devices 170 incorporating multiple instances of one or more sensors 130. In some cases, multiple iterations of system 100 can be used to monitor multiple members of a sleep cohort (e.g., a separate system 100 for each member of the cohort). Aspects and features of system 100 can be used to monitor sleep and interact with any members of a cohort, such as a therapy user and a therapy-adjacent user.

The control system 110 includes one or more processors 112 (hereinafter, processor 112). The control system 110 is generally used to control (e.g., actuate) the various components of the system 100 and/or analyze data obtained and/or generated by the components of the system 100. The processor 112 can be a general or special purpose processor or microprocessor. While one processor 112 is shown in FIG. 1, the control system 110 can include any suitable number of processors (e.g., one processor, two processors, five processors, ten processors, etc.) that can be in a single housing, or located remotely from each other. The control system 110 (or any other control system) or a portion of the control system 110 such as the processor 112 (or any other processor(s) or portion(s) of any other control system), can be used to carry out one or more steps of any of the methods described and/or claimed herein. The control system 110 can be coupled to and/or positioned within, for example, a housing of the user device 170, a portion (e.g., a housing) of the respiratory system 120, and/or within a housing of one or more of the sensors 130. The control system 110 can be centralized (within one such housing) or decentralized (within two or more of such housings, which are physically distinct). In such implementations including two or more housings containing the control system 110, such housings can be located proximately and/or remotely from each other.

The memory device 114 stores machine-readable instructions that are executable by the processor 112 of the control system 110. The memory device 114 can be any suitable computer readable storage device or media, such as, for example, a random or serial access memory device, a hard drive, a solid state drive, a flash memory device, etc. While one memory device 114 is shown in FIG. 1, the system 100 can include any suitable number of memory devices 114 (e.g., one memory device, two memory devices, five memory devices, ten memory devices, etc.). The memory device 114 can be coupled to and/or positioned within a housing of the respiratory device 122, within a housing of the user device 170, within a housing of one or more of the sensors 130, or any combination thereof. Like the control system 110, the memory device 114 can be centralized (within one such housing) or decentralized (within two or more of such housings, which are physically distinct).

In some implementations, the memory device 114 (FIG. 1) stores a member profile associated with a member of the cohort. The member profile can include an identification of the sleep cohort to which the member is a member. The member profile can include, for example, demographic information associated with the cohort member, biometric information associated with the cohort member, medical information associated with the cohort member, self-reported feedback, sleep parameters associated with the cohort member (e.g., sleep-related parameters recorded from one or more earlier sleep sessions), or any combination thereof. The demographic information can include, for example, information indicative of an age of the cohort member, a gender of the cohort member, a race of the cohort member, a geographic location of the cohort member, a relationship status, a family history of insomnia, an employment status of the cohort member, an educational status of the cohort member, a socioeconomic status of the cohort member, or any combination thereof. The medical information can include, for example, including indicative of one or more medical conditions associated with the cohort member, medication usage by the cohort member, or both. The medical information data can further include fall risk assessment associated with the user (e.g., a fall risk score using the Morse fall scale), a multiple sleep latency test (MSLT) test result or score and/or a Pittsburgh Sleep Quality Index (PSQI) score or value. The self-reported feedback can include information indicative of a self-reported subjective sleep score (e.g., poor, average, excellent), a self-reported subjective stress level of the cohort member, a self-reported subjective fatigue level of the cohort member, a self-reported subjective health status of the cohort member, a recent life event experienced by the cohort member, or any combination thereof.

The electronic interface 119 is configured to receive data (e.g., physiological data and/or audio data) from the one or more sensors 130 such that the data can be stored in the memory device 114 and/or analyzed by the processor 112 of the control system 110. The electronic interface 119 can communicate with the one or more sensors 130 using a wired connection or a wireless connection (e.g., using an RF communication protocol, a WiFi communication protocol, a Bluetooth communication protocol, over a cellular network, etc.). The electronic interface 119 can include an antenna, a receiver (e.g., an RF receiver), a transmitter (e.g., an RF transmitter), a transceiver, or any combination thereof. The electronic interface 119 can also include one more processors and/or one more memory devices that are the same as, or similar to, the processor 112 and the memory device 114 described herein. In some implementations, the electronic interface 119 is coupled to or integrated in the user device 170. In other implementations, the electronic interface 119 is coupled to or integrated (e.g., in a housing) with the control system 110 and/or the memory device 114.

As noted above, in some implementations, the system 100 optionally includes a respiratory system 120 (also referred to as a respiratory therapy system). The respiratory system 120 can include a respiratory pressure therapy device 122 (referred to herein as respiratory device 122), a user interface 124 (also referred to as a mask or a patient interface), a conduit 126 (also referred to as a tube or an air circuit), a display device 128, a humidification tank 129, or any combination thereof. In some implementations, the control system 110, the memory device 114, the display device 128, one or more of the sensors 130, and the humidification tank 129 are part of the respiratory device 122. Respiratory pressure therapy refers to the application of a supply of air to an entrance to a therapy user's airways at a controlled target pressure that is nominally positive with respect to atmosphere throughout the therapy user's breathing cycle (e.g., in contrast to negative pressure therapies such as the tank ventilator or cuirass). The respiratory system 120 is generally used to treat individuals suffering from one or more sleep-related respiratory disorders (e.g., obstructive sleep apnea, central sleep apnea, or mixed sleep apnea).

The respiratory device 122 is generally used to generate pressurized air that is delivered to a therapy user (e.g., using one or more motors (such as a blower motor) that drive one or more compressors). In some implementations, the respiratory device 122 generates continuous constant air pressure that is delivered to the therapy user. In other implementations, the respiratory device 122 generates two or more predetermined pressures (e.g., a first predetermined air pressure and a second predetermined air pressure). In still other implementations, the respiratory device 122 is configured to generate a variety of different air pressures within a predetermined range. For example, the respiratory device 122 can deliver at least about 6 cm H₂O, at least about 10 cm H₂O, at least about 20 cm H₂O, between about 6 cm H₂O and about 10 cm H₂O, between about 7 cm H₂O and about 12 cm H₂O, etc. The respiratory device 122 can also deliver pressurized air at a predetermined flow rate between, for example, about −20 L/min and about 150 L/min, while maintaining a positive pressure (relative to the ambient pressure).

The user interface 124 engages a portion of the therapy user's face and delivers pressurized air from the respiratory device 122 to the therapy user's airway to aid in preventing the airway from narrowing and/or collapsing during sleep. This may also increase the therapy user's oxygen intake during sleep. Depending upon the therapy to be applied, the user interface 124 may form a seal, for example, with a region or portion of the therapy user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cm H₂O.

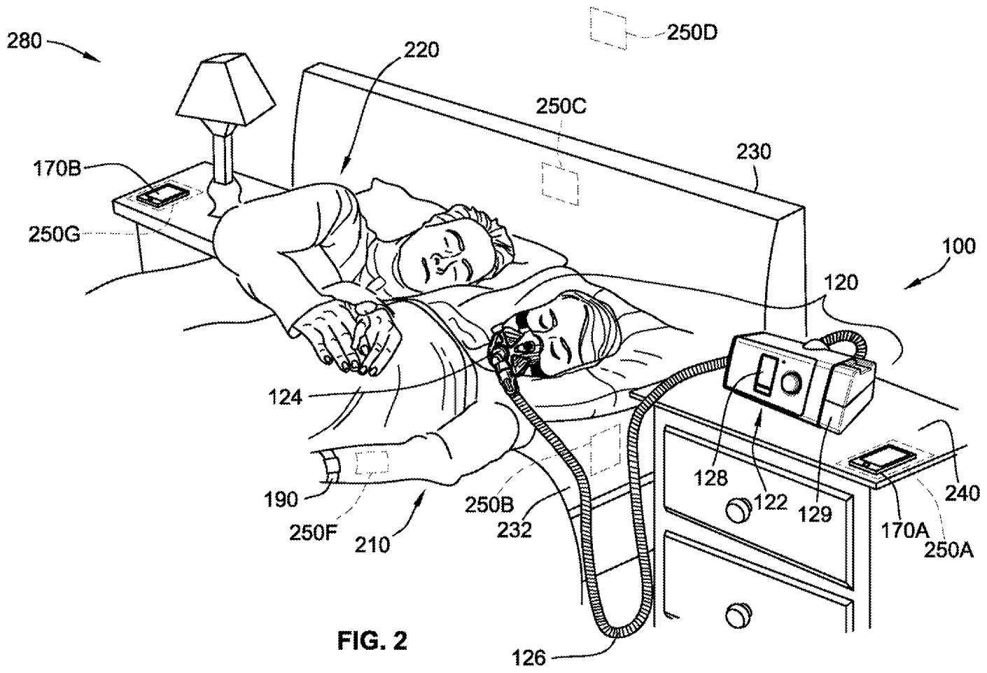

As shown in FIG. 2, in some implementations, the user interface 124 is a face mask that covers the nose and mouth of the therapy user (as shown, for example, in FIG. 2). Alternatively, the user interface 124 can be a nasal mask that provides air to the nose of the therapy user or a nasal pillow mask that delivers air directly to the nostrils of the therapy user. The user interface 124 can include a plurality of straps (e.g., including hook and loop fasteners) for positioning and/or stabilizing the interface on a portion of the therapy user (e.g., the face) and a conformal cushion (e.g., silicone, plastic, foam, etc.) that aids in providing an air-tight seal between the user interface 124 and the therapy user. In some examples, the user interface 124 can be a tube-up mask, wherein straps of the mask are configured to act as conduit(s) to deliver pressurized air to the face or nasal mask. The user interface 124 can also include one or more vents for permitting the escape of carbon dioxide and other gases exhaled by the therapy user 210. In other implementations, the user interface 124 can comprise a mouthpiece (e.g., a night guard mouthpiece molded to conform to the therapy user's teeth, a mandibular repositioning device, etc.).

The conduit 126 (also referred to as an air circuit or tube) allows the flow of air between two components of a respiratory system 120, such as the respiratory device 122 and the user interface 124. In some implementations, there can be separate limbs of the conduit for inhalation and exhalation. In other implementations, a single limb conduit is used for both inhalation and exhalation. Generally, the respiratory therapy system 120 forms an air pathway that extends between a motor of the respiratory therapy device 122 and the user and/or the user's airway. Thus, the air pathway generally includes at least a motor of the respiratory therapy device 122, the user interface 124, and the conduit 126.

One or more of the respiratory device 122, the user interface 124, the conduit 126, the display device 128, and the humidification tank 129 can contain one or more sensors (e.g., a pressure sensor, a flow rate sensor, or more generally any of the other sensors 130 described herein). These one or more sensors can be use, for example, to measure the air pressure and/or flow rate of pressurized air supplied by the respiratory device 122.

The display device 128 is generally used to display image(s) including still images, video images, or both and/or information regarding the respiratory device 122. For example, the display device 128 can provide information regarding the status of the respiratory device 122 (e.g., whether the respiratory device 122 is on/off, the pressure of the air being delivered by the respiratory device 122, the temperature of the air being delivered by the respiratory device 122, etc.) and/or other information (e.g., sleep performance metrics, a sleep performance score, a sleep score or a therapy score (such as a myAir™ score, such as described in WO 2016/061629 and US 2017/0311879, each of which is hereby incorporated by reference herein in its entirety), the current date/time, personal information for the therapy user, questionnaire for the user, etc.). In some implementations, the display device 128 acts as a human-machine interface (HMI) that includes a graphic user interface (GUI) configured to display the image(s) as an input interface. The display device 128 can be an LED display, an OLED display, an LCD display, or the like. The input interface can be, for example, a touchscreen or touch-sensitive substrate, a mouse, a keyboard, or any sensor system configured to sense inputs made by a human individual interacting with the respiratory device 122.

The humidification tank 129 is coupled to or integrated in the respiratory device 122 and includes a reservoir of water that can be used to humidify the pressurized air delivered from the respiratory device 122. The respiratory device 122 can include a heater to heat the water in the humidification tank 129 in order to humidify the pressurized air provided to the therapy user. Additionally, in some implementations, the conduit 126 can also include a heating element (e.g., coupled to and/or imbedded in the conduit 126) that heats the pressurized air delivered to the therapy user. The humidification tank 129 can be fluidly coupled to a water vapor inlet of the air pathway and deliver water vapor into the air pathway via the water vapor inlet, or can be formed in-line with the air pathway as part of the air pathway itself. In other implementations, the respiratory therapy device 122 or the conduit 126 can include a waterless humidifier. The waterless humidifier can incorporate sensors that interface with other sensor positioned elsewhere in system 100.

The respiratory system 120 can be used, for example, as a ventilator or a positive airway pressure (PAP) system such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the therapy user. The APAP system automatically varies the air pressure delivered to the therapy user based on, for example, respiration data associated with the therapy user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

Referring to FIG. 2, a portion of the system 100 (FIG. 1), according to some implementations, is illustrated. A therapy user 210 of the respiratory system 120 and a therapy-adjacent individual 220 (e.g., a bed partner) are located in a bed 230 and are laying on a mattress 232 within an environment 280. The user interface 124 (e.g., a full face mask) can be worn by the therapy user 210 during the therapy user's sleep session. The user interface 124 is fluidly coupled and/or connected to the respiratory device 122 via the conduit 126. In turn, the respiratory device 122 delivers pressurized air to the therapy user 210 via the conduit 126 and the user interface 124 to increase the air pressure in the throat of the therapy user 210 to aid in preventing the airway from closing and/or narrowing during sleep. The respiratory therapy device 122 can include the display device 128, which can allow the user to interact with the respiratory therapy device 122. The respiratory therapy device 122 can also include the humidification tank 129, which stores the water used to humidify the pressurized air. The respiratory therapy device 122 can be positioned on a nightstand 240 that is directly adjacent to the bed 230 as shown in FIG. 2, or more generally, on any surface or structure that is generally adjacent to the bed 230 and/or the user 210. The user can also wear, for example, a blood pressure device and/or activity tracker 190 while lying on the mattress 232 in the bed 230.

As depicted in FIG. 2, the therapy user 210 can have a user device 170A and the therapy-adjacent individual 220 can have their own user device 170B, although that need not always be the case. User device 170A, 170B can be iterations of user device 170, and can each include any combination of one or more sensors 130 used to obtain sensor data usable to generate sleep performance metrics as disclosed herein.

Referring to back to FIG. 1, the one or more sensors 130 of the system 100 include a pressure sensor 132, a flow rate sensor 134, temperature sensor 136, a motion sensor 138, a microphone 140, a speaker 142, a radio-frequency (RF) receiver 146, a RF transmitter 148, a camera 150, an infrared sensor 152, a photoplethysmogram (PPG) sensor 154, an electrocardiogram (ECG) sensor 156, an electroencephalography (EEG) sensor 158, a capacitive sensor 160, a force sensor 162, a strain gauge sensor 164, an electromyography (EMG) sensor 166, an oxygen sensor 168, an analyte sensor 174, a moisture sensor 176, a LiDAR sensor 178, or any combination thereof. Generally, each of the one or sensors 130 are configured to output sensor data that is received and stored in the memory device 114 or one or more other memory devices.

While the one or more sensors 130 are shown and described as including each of the pressure sensor 132, the flow rate sensor 134, the temperature sensor 136, the motion sensor 138, the microphone 140, the speaker 142, the RF receiver 146, the RF transmitter 148, the camera 150, the infrared sensor 152, the photoplethysmogram (PPG) sensor 154, the electrocardiogram (ECG) sensor 156, the electroencephalography (EEG) sensor 158, the capacitive sensor 160, the force sensor 162, the strain gauge sensor 164, the electromyography (EMG) sensor 166, the oxygen sensor 168, the analyte sensor 174, the moisture sensor 176, and the LiDAR sensor 178, more generally, the one or more sensors 130 can include any combination and any number of each of the sensors described and/or shown herein.

The one or more sensors 130 can be used to generate sensor data, such as physiological data, audio data, or both. Physiological data generated by one or more of the sensors 130 can be used by the control system 110 to determine a sleep-wake signal associated with a cohort member during a sleep session and one or more sleep-related parameters. The sleep-wake signal can be indicative of one or more sleep states, including wakefulness, relaxed wakefulness, micro-awakenings, a rapid eye movement (REM) stage, a first non-REM stage (often referred to as “N1”), a second non-REM stage (often referred to as “N2”), a third non-REM stage (often referred to as “N3”), or any combination thereof. N1 and N2 can be considered light sleep stages, whereas N3 can be considered a deep sleep stage. Methods for determining sleep stages from physiological data generated by one or more of the sensors, such as sensors 130, are described in, for example, WO 2014/047310, U.S. Pat. Nos. 10,492,720, 10,660,563, US 2020/0337634, WO 2017/132726, WO 2019/122413, US 2021/0150873, WO 2019/122414, US 2020/0383580, each of which is hereby incorporated by reference herein in its entirety. The sleep-wake signal can also be timestamped to indicate a time that the cohort member enters the bed, a time that the cohort member exits the bed, a time that the cohort member attempts to fall asleep, etc. The sleep-wake signal can be measured by the sensor(s) 130 during the sleep session at a predetermined sampling rate, such as, for example, one sample per second, one sample per 30 seconds, one sample per minute, etc. Examples of the one or more sleep-related parameters that can be determined for the cohort member during the sleep session based on the sleep-wake signal include a total time in bed, a total sleep time, a sleep onset latency, a wake-after-sleep-onset parameter, a sleep efficiency, a fragmentation index, or any combination thereof.

Physiological data and/or audio data generated by the one or more sensors 130 can also be used to determine a respiration signal associated with a cohort member during a sleep session. The respiration signal is generally indicative of respiration or breathing of the cohort member during the sleep session. The respiration signal can be indicative of, for example, a respiration rate, a respiration rate variability, an inspiration amplitude, an expiration amplitude, an inspiration-expiration ratio, a number of events per hour, a pattern of events, pressure settings of the respiratory device 122, or any combination thereof. The event(s) can include snoring, apneas, central apneas, obstructive apneas, mixed apneas, hypopneas, RERAs, a flow limitation (e.g., an event that results in the absence of the increase in flow despite an elevation in negative intrathoracic pressure indicating increased effort), a mask leak (e.g., from the user interface 124), a restless leg, a sleeping disorder, choking, an increased heart rate, labored breathing, an asthma attack, an epileptic episode, a seizure, increased blood pressure, hyperventilation, or any combination thereof. Events can be detected by any means known in the art such as described in, for example, U.S. Pat. Nos. 5,245,995, 6,502,572, WO 2018/050913, WO 2020/104465, each of which is incorporated by reference herein in its entirety.

The pressure sensor 132 outputs pressure data that can be stored in the memory device 114 and/or analyzed by the processor 112 of the control system 110. In some implementations, the pressure sensor 132 is an air pressure sensor (e.g., barometric pressure sensor) that generates sensor data indicative of the respiration (e.g., inhaling and/or exhaling) of the therapy user using the respiratory system 120 and/or ambient pressure. In such implementations, the pressure sensor 132 can be coupled to or integrated in the respiratory device 122. The pressure sensor 132 can be, for example, a capacitive sensor, an electromagnetic sensor, a piezoelectric sensor, a strain-gauge sensor, an optical sensor, a potentiometric sensor, or any combination thereof. In one example, the pressure sensor 132 can be used to determine a blood pressure of a cohort member.

The flow rate sensor 134 outputs flow rate data that can be stored in the memory device 114 and/or analyzed by the processor 112 of the control system 110. In some implementations, the flow rate sensor 134 is used to determine an air flow rate from the respiratory device 122, an air flow rate through the conduit 126, an air flow rate through the user interface 124, or any combination thereof. In such implementations, the flow rate sensor 134 can be coupled to or integrated in the respiratory device 122, the user interface 124, or the conduit 126. The flow rate sensor 134 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof.

The temperature sensor 136 outputs temperature data that can be stored in the memory device 114 and/or analyzed by the processor 112 of the control system 110. In some implementations, the temperature sensor 136 generates temperatures data indicative of a core body temperature of a cohort member, a skin temperature of a cohort member, a temperature of the air flowing from the respiratory device 122 and/or through the conduit 126, a temperature in the user interface 124, an ambient temperature, or any combination thereof. The temperature sensor 136 can be, for example, a thermocouple sensor, a thermistor sensor, a silicon band gap temperature sensor or semiconductor-based sensor, a resistance temperature detector, or any combination thereof.

The motion sensor 138 outputs motion data that can be stored in the memory device 114 and/or analyzed by the processor 112 of the control system 110. The motion sensor 138 can be used to detect movement of the user during the sleep session, and/or detect movement of any of the components of the respiratory therapy system 120, such as the respiratory therapy device 122, the user interface 124, or the conduit 126. The motion sensor 138 can include one or more inertial sensors, such as accelerometers, gyroscopes, and magnetometers. The motion sensor 138 can be used to detect motion or acceleration associated with arterial pulses, such as pulses in or around the face of the user and proximal to the user interface 124, and configured to detect features of the pulse shape, speed, amplitude, or volume. In some implementations, the motion sensor 138 alternatively or additionally generates one or more signals representing bodily movement of the user, from which may be obtained a signal representing a sleep state of the user; for example, via a respiratory movement of the user.

The microphone 140 outputs audio data that can be stored in the memory device 114 and/or analyzed by the processor 112 of the control system 110. The audio data generated by the microphone 140 is reproducible as one or more sound(s) during a sleep session (e.g., sounds from a cohort member such as the therapy user 210 and/or the therapy-adjacent individual 220). The audio data form the microphone 140 can also be used to identify (e.g., using the control system 110) an event experienced by the cohort member during the sleep session, as described in further detail herein. The microphone 140 can be coupled to or integrated in the respiratory device 122, the use interface 124, the conduit 126, the user device 170A, or the user device 170B. For example, the microphone 140 can be disposed inside the respiratory therapy device 122, the user interface 124, the conduit 126, or other components. The microphone 140 can also be positioned adjacent to or coupled to the outside of the respiratory therapy device 122, the outside of the user interface 124, the outside of the conduit 126, or outside of any other components. The microphone 140 could also be a component of the user device 170 (e.g., the microphone 140 is a microphone of a smart phone). The microphone 140 can be integrated into the user interface 124, the conduit 126, the respiratory therapy device 122, or any combination thereof. In general, the microphone 140 can be located at any point within or adjacent to the air pathway of the respiratory therapy system 120, which includes at least the motor of the respiratory therapy device 122, the user interface 124, and the conduit 126. Thus, the air pathway can also be referred to as the acoustic pathway.

The speaker 142 outputs sound waves that are typically audible to a cohort member using the system 100 (e.g., the therapy user 210 of FIG. 2). In one or more implementations, the sound waves can be audible to a user of the system 100 or inaudible to the user of the system (e.g., ultrasonic sound waves). The speaker 142 can be used, for example, as an alarm clock or to play an alert or message to the cohort member (e.g., in response to an event). In some implementations, the speaker 142 can be used to communicate the audio data generated by the microphone 140 to the cohort member. The speaker 142 can be coupled to or integrated in the respiratory device 122, the user interface 124, the conduit 126, the user device 170A, or the user device 170B.

The microphone 140 and the speaker 142 can be used as separate devices. In some implementations, the microphone 140 and the speaker 142 can be combined into an acoustic sensor 141, as described in, for example, WO 2018/050913, which is hereby incorporated by reference herein in its entirety. In such implementations, the speaker 142 generates or emits sound waves at a predetermined interval and the microphone 140 detects the reflections of the emitted sound waves from the speaker 142. The sound waves generated or emitted by the speaker 142 have a frequency that is not audible to the human ear (e.g., below 20 Hz or above around 18 kHz) so as not to disturb the sleep of the therapy user 210 or the therapy-adjacent individual 220 (FIG. 2). Based at least in part on the data from the microphone 140 and/or the speaker 142, the control system 110 can determine a location of the therapy user 210 or therapy-adjacent individual 220 (FIG. 2) and/or one or more of the sleep-related parameters described in herein, such as, for example, a respiration signal, a respiration rate, an inspiration amplitude, an expiration amplitude, an inspiration-expiration ratio, a number of events per hour, a pattern of events, a sleep stage, pressure settings of the respiratory therapy device 122, a mouth leak status, or any combination thereof. In this context, a SONAR sensor may be understood to concern an active acoustic sensing, such as by generating/transmitting ultrasound or low frequency ultrasound sensing signals (e.g., in a frequency range of about 17-23 kHz, 18-22 kHz, or 17-18 kHz, for example), through the air. Such a system may be considered in relation to WO 2018/050913 and WO 2020/104465 mentioned above. In some implementations, the speaker 142 is a bone conduction speaker. In some implementations, the one or more sensors 130 include (i) a first microphone that is the same or similar to the microphone 140, and is integrated into the acoustic sensor 141 and (ii) a second microphone that is the same as or similar to the microphone 140, but is separate and distinct from the first microphone that is integrated into the acoustic sensor 141.

In some implementations, the sensors 130 include (i) a first microphone that is the same as, or similar to, the microphone 140, and is integrated in the acoustic sensor 141 and (ii) a second microphone that is the same as, or similar to, the microphone 140, but is separate and distinct from the first microphone that is integrated in the acoustic sensor 141.

The RF transmitter 148 generates and/or emits radio waves having a predetermined frequency and/or a predetermined amplitude (e.g., within a high frequency band, within a low frequency band, long wave signals, short wave signals, etc.). The RF receiver 146 detects the reflections of the radio waves emitted from the RF transmitter 148, and this data can be analyzed by the control system 110 to determine a location of a cohort member and/or one or more of the sleep-related parameters described herein. An RF receiver (either the RF receiver 146 and the RF transmitter 148 or another RF pair) can also be used for wireless communication between the control system 110, the respiratory device 122, the one or more sensors 130, the user device 170A, the user device 170B, or any combination thereof. While the RF receiver 146 and RF transmitter 148 are shown as being separate and distinct elements in FIG. 1, in some implementations, the RF receiver 146 and RF transmitter 148 are combined as a part of an RF sensor 147. In some such implementations, the RF sensor 147 includes a control circuit. The specific format of the RF communication can be WiFi, Bluetooth, or the like.

In some implementations, the RF sensor 147 is a part of a mesh system. One example of a mesh system is a WiFi mesh system, which can include mesh nodes, mesh router(s), and mesh gateway(s), each of which can be mobile/movable or fixed. In such implementations, the WiFi mesh system includes a WiFi router and/or a WiFi controller and one or more satellites (e.g., access points), each of which include an RF sensor that the is the same as, or similar to, the RF sensor 147. The WiFi router and satellites continuously communicate with one another using WiFi signals. The WiFi mesh system can be used to generate motion data based on changes in the WiFi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 150 outputs image data reproducible as one or more images (e.g., still images, video images, thermal images, or a combination thereof) that can be stored in the memory device 114. The image data from the camera 150 can be used by the control system 110 to determine one or more of the sleep-related parameters described herein. For example, the image data from the camera 150 can be used to identify a location of a cohort member, to determine a time when the cohort member enters the bed, and to determine a time when the cohort member exits the bed.

The infrared (IR) sensor 152 outputs infrared image data reproducible as one or more infrared images (e.g., still images, video images, or both) that can be stored in the memory device 114. The infrared data from the IR sensor 152 can be used to determine one or more sleep-related parameters during a sleep session, including a temperature of the cohort member and/or movement of the cohort member. The IR sensor 152 can also be used in conjunction with the camera 150 when measuring the presence, location, and/or movement of the cohort member. The IR sensor 152 can detect infrared light having a wavelength between about 700 nm and about 1 mm, for example, while the camera 150 can detect visible light having a wavelength between about 380 nm and about 740 nm.

The PPG sensor 154 outputs physiological data associated with the cohort member that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 154 can be worn by the cohort member, embedded in clothing and/or fabric that is worn by the cohort member, embedded in and/or coupled to the user interface 124 and/or its associated headgear (e.g., straps, etc.), etc.

The ECG sensor 156 outputs physiological data associated with electrical activity of the heart of the cohort member. In some implementations, the ECG sensor 156 includes one or more electrodes that are positioned on or around a portion of the cohort member during the sleep session. The physiological data from the ECG sensor 156 can be used, for example, to determine one or more of the sleep-related parameters described herein.

The EEG sensor 158 outputs physiological data associated with electrical activity of the brain of the cohort member. In some implementations, the EEG sensor 158 includes one or more electrodes that are positioned on or around the scalp of the cohort member during the sleep session. The physiological data from the EEG sensor 158 can be used, for example, to determine a sleep state of the cohort member at any given time during the sleep session. In some implementations, the EEG sensor 158 can be integrated in the user interface 124 and/or the associated headgear (e.g., straps, etc.).

The capacitive sensor 160, the force sensor 162, and the strain gauge sensor 164 output data that can be stored in the memory device 114 and used by the control system 110 to determine one or more of the sleep-related parameters described herein. The EMG sensor 166 outputs physiological data associated with electrical activity produced by one or more muscles. The oxygen sensor 168 outputs oxygen data indicative of an oxygen concentration of gas (e.g., in the conduit 126 or at the user interface 124). The oxygen sensor 168 can be, for example, an ultrasonic oxygen sensor, an electrical oxygen sensor, a chemical oxygen sensor, an optical oxygen sensor, or any combination thereof. In some implementations, the one or more sensors 130 also include a galvanic skin response (GSR) sensor, a blood flow sensor, a respiration sensor, a pulse sensor, a sphygmomanometer sensor, an oximetry sensor, or any combination thereof.

The analyte sensor 174 can be used to detect the presence of an analyte in the exhaled breath of the cohort member (e.g., therapy user 210). The data output by the analyte sensor 174 can be stored in the memory device 114 and used by the control system 110 to determine the identity and concentration of any analytes in the breath of the therapy user 210. In some implementations, the analyte sensor 174 is positioned near a mouth of the therapy user 210 to detect analytes in breath exhaled from the therapy user 210's mouth. For example, when the user interface 124 is a face mask that covers the nose and mouth of the therapy user 210, the analyte sensor 174 can be positioned within the face mask to monitor the therapy user 210's mouth breathing. In other implementations, such as when the user interface 124 is a nasal mask or a nasal pillow mask, the analyte sensor 174 can be positioned near the nose of the therapy user 210 to detect analytes in breath exhaled through the therapy user's nose. In still other implementations, the analyte sensor 174 can be positioned near the therapy user 210's mouth when the user interface 124 is a nasal mask or a nasal pillow mask. In this implementation, the analyte sensor 174 can be used to detect whether any air is inadvertently leaking from the therapy user 210's mouth. In some implementations, the analyte sensor 174 is a volatile organic compound (VOC) sensor that can be used to detect carbon-based chemicals or compounds. In some implementations, the analyte sensor 174 can also be used to detect whether the therapy user 210 is breathing through their nose or mouth. For example, if the data output by an analyte sensor 174 positioned near the mouth of the therapy user 210 or within the face mask (in implementations where the user interface 124 is a face mask) detects the presence of an analyte, the control system 110 can use this data as an indication that the therapy user 210 is breathing through their mouth.

The moisture sensor 176 outputs data that can be stored in the memory device 114 and used by the control system 110. The moisture sensor 176 can be used to detect moisture in various areas surrounding the therapy user (e.g., inside the conduit 126 or the user interface 124, near the therapy user 210's face, near the connection between the conduit 126 and the user interface 124, near the connection between the conduit 126 and the respiratory device 122, etc.). Thus, in some implementations, the moisture sensor 176 can be coupled to or integrated in the user interface 124 or in the conduit 126 to monitor the humidity of the pressurized air from the respiratory device 122. In other implementations, the moisture sensor 176 is placed near any area where moisture levels need to be monitored. The moisture sensor 176 can also be used to monitor the ambient humidity of the environment 280 surrounding the therapy user 210 and/or the therapy-adjacent individual 220, for example, the air inside the bedroom.

The Light Detection and Ranging (LiDAR) sensor 178 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 166 can measure and map an area extending 5 meters or more away from the sensor. The LiDAR data can be fused with point cloud data estimated by an electromagnetic RADAR sensor, for example. The LiDAR sensor(s) 178 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

While shown separately in FIG. 1, any combination of the one or more sensors 130 can be integrated in and/or coupled to any one or more of the components of the system 100, including the respiratory device 122, the user interface 124, the conduit 126, the humidification tank 129, the control system 110, the user device 170 (e.g., user devices 170A, 170B of FIG. 2), or any combination thereof. For example, the microphone 140 and speaker 142 is integrated in and/or coupled to the user device 170 and the pressure sensor 130 and/or flow rate sensor 132 are integrated in and/or coupled to the respiratory device 122. In some implementations, at least one of the one or more sensors 130 is not coupled to the respiratory device 122, the control system 110, or the user device 170, and is positioned generally adjacent to the therapy user 210 or therapy-adjacent individual 220 during the sleep session (e.g., positioned on or in contact with a portion of the therapy user 210 or therapy-adjacent individual 220, worn by the therapy user 210 or therapy—adjacent individual 220, coupled to or positioned on the nightstand, coupled to the mattress, coupled to the ceiling, etc.).

For example, as shown in FIG. 2, one or more of the sensors 130 can be located in a first position 250A on the nightstand 240 adjacent to the bed 230 and the therapy user 210. Alternatively, one or more of the sensors 130 can be located in a second position 250B on and/or in the mattress 232 (e.g., the sensor is coupled to and/or integrated in the mattress 232). Further, one or more of the sensors 130 can be located in a third position 250C on the bed 230 (e.g., the secondary sensor(s) 140 is couple to and/or integrated in a headboard, a footboard, or other location on the frame of the bed 230). One or more of the sensors 130 can also be located in a fourth position 250D on a wall or ceiling that is generally adjacent to the bed 230 and/or the user 210. The one or more of the sensors 130 can also be located in a fifth position such that the one or more of the sensors 130 is coupled to and/or positioned on and/or inside a housing of the respiratory device 122 of the respiratory system 120. Further, one or more of the sensors 130 can be located in a sixth position 250F such that the sensor is coupled to and/or positioned on the therapy user 210 (e.g., the sensor(s) is embedded in or coupled to fabric or clothing worn by the therapy user 210 during the sleep session). Likewise, one or more of the sensors 130 can be located in a seventh position such that the sensor is coupled to and/or positioned on the therapy-adjacent individual 220 (e.g., the sensor(s) is embedded in or coupled to fabric or clothing worn by the therapy-adjacent individual 220 during the sleep session). Further, one or more of the sensors 130 can eb located in a eight position 250G on the nightstand adjacent to bed 230 and the therapy-adjacent individual 220. More generally, the one or more of the sensors 130 can be positioned at any suitable location relative to the cohort member being monitored such that the sensor(s) 140 can generate physiological data associated with the cohort member (e.g., the therapy user 210 and/or the therapy-adjacent individual 220) during one or more sleep session.

The user device 170 (FIG. 1) includes a display device 172. The user device 170 can be, for example, a mobile device such as a smart phone, a tablet, a laptop, or the like. Alternatively, the user device 170 can be an external sensing system, a television (e.g., a smart television) or another smart home device (e.g., a smart speaker(s) such as Google Home™ Google Nest™, Amazon Echo™, Amazon Echo Show™, Alexa™-enabled devices, etc.). In some implementations, the user device is a wearable device (e.g., a smart watch). The display device 172 is generally used to display image(s) including still images, video images, or both. In some implementations, the display device 172 acts as a human-machine interface (HMI) that includes a graphic user interface (GUI) configured to display the image(s) and an input interface. The display device 172 can be an LED display, an OLED display, an LCD display, or the like. The input interface can be, for example, a touchscreen or touch-sensitive substrate, a mouse, a keyboard, or any sensor system configured to sense inputs made by a human individual interacting with the user device 170. In some implementations, one or more user devices can be used by and/or included in the system 100, such as a separate user device for each member of the sleep cohort.

The light source 180 is generally used to emit light having an intensity and a wavelength (e.g., color). For example, the light source 180 can emit light having a wavelength between about 380 nm and about 700 nm (e.g., a wavelength in the visible light spectrum). The light source 180 can include, for example, one or more light emitting diodes (LEDs), one or more organic light emitting diodes (OLEDs), a light bulb, a lamp, an incandescent light bulb, a CFL lightbulb, a halogen lightbulb, or any combination thereof. In some implementations, the intensity and/or wavelength (e.g., color) of light emitted from the light source 180 can be modified by the control system 110. The light source 180 can also emit light in a predetermined pattern of emission, such as, for example, continuous emission, pulsed emission, periodic emission of differing intensities (e.g., light emission cycles including a gradual increase in intensity followed by a decrease in intensity), or any combination thereof. Light emitted from the light source 180 can be viewed directly by the cohort member or, alternatively, reflected or refracted prior to reaching the cohort member. In some implementations, the light source 180 includes one or more light pipes.

In some implementations, the light source 180 is physically coupled to or integrated in the respiratory therapy system 120. For example, the light source 180 can be physically coupled to or integrated in the respiratory device 122, the user interface 124, the conduit 126, the display device 128, or any combination thereof. In some implementations, the light source 180 is physically coupled to or integrated in the user device 170. In other implementations, the light source 180 is separate and distinct from each of the respiratory therapy system 120 and the user device 170, and the activity tracker 190. In such implementations, the light source 180 can be positioned towards the cohort member, for example, on the nightstand 240, the bed 230, other furniture, a wall, a ceiling, etc.

The activity tracker 190 is generally used to aid in generating physiological data for determining an activity measurement associated with the cohort member (e.g., therapy user 210 or therapy-adjacent individual 220). The activity measurement can include, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. The activity tracker 190 includes one or more of the sensors 130 described herein, such as, for example, the motion sensor 138 (e.g., one or more accelerometers and/or gyroscopes), the PPG sensor 154, and/or the ECG sensor 156.

In some implementations, the activity tracker 190 is a wearable device that can be worn by the cohort member, such as a smartwatch, a wristband, a ring, or a patch. For example, referring to FIG. 2, the activity tracker 190 is worn on a wrist of the therapy user 210. The activity tracker 190 can also be coupled to or integrated into a garment or clothing that is worn by the therapy user. In some cases, a similar activity tracker can be worn on the wrist of the therapy-adjacent individual 220 or coupled to or integrated into a garment or clothing that is worn by the therapy-adjacent individual 220. Alternatively still, the activity tracker 190 can also be coupled to or integrated in (e.g., within the same housing) user device 170A and/or user device 170B. More generally, the activity tracker 190 can be communicatively coupled with, or physically integrated in (e.g., within a housing), the control system 110, the memory 114, the respiratory system 120, the user device 170A, and/or the user device 170B.

Referring back to FIG. 1, while the control system 110 and the memory device 114 are described and shown in FIG. 1 as being a separate and distinct component of the system 100, in some implementations, the control system 110 and/or the memory device 114 are integrated in the user device 170 and/or the respiratory device 122. Alternatively, in some implementations, the control system 110 or a portion thereof (e.g., the processor 112) can be located in a cloud (e.g., integrated in a server, integrated in an Internet of Things (IoT) device (e.g., a smart TV, a smart thermostat, a smart appliance, smart lighting, etc.), connected to the cloud, be subject to edge cloud processing, etc.), located in one or more servers (e.g., remote servers, local servers, etc., or any combination thereof.

While system 100 is shown as including all of the components described above, more or fewer components can be included in a system for generating physiological data and determining a recommended notification or action for the cohort member according to implementations of the present disclosure. For example, a first alternative system includes the control system 110, the memory device 114, and at least one of the one or more sensors 130. As another example, a second alternative system includes the control system 110, the memory device 114, at least one of the one or more sensors 130, and the user device 170. As yet another example, a third alternative system includes the control system 110, the memory device 114, the respiratory system 120, at least one of the one or more sensors 130, and first and second user devices 170. Thus, various systems can be formed using any portion or portions of the components shown and described herein and/or in combination with one or more other components.

As used herein, a sleep session can be defined in a number of ways based on, for example, an initial start time and an end time. Referring to FIG. 3, an exemplary timeline 301 for a sleep session is illustrated. The timeline 301 includes an enter bed time (t_{bed}), a go-to-sleep time (t_{GTS}), an initial sleep time tₛₗₑₑₚ, a first micro-awakening MA₁ and a second micro-awakening MA₂, a wake-up time (t_{wake}), and a rising time (tᵣᵢₛₑ).

In some implementations, a sleep session is a duration where the cohort member is asleep. In such implementations, the sleep session has a start time and an end time, and during the sleep session, the cohort member does not wake until the end time. That is, any period of the cohort member being awake is not included in a sleep session. From this first definition of sleep session, if the cohort member wakes ups and falls asleep multiple times in the same night, each of the sleep intervals separated by an awake interval is a sleep session.

Alternatively, in some implementations, a sleep session has a start time and an end time, and during the sleep session, the cohort member can wake up, without the sleep session ending, so long as a continuous duration that the cohort member is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the cohort member first entered the bed, and the time the next morning when cohort member last left the bed. Put another way, a sleep session can be defined as a period of time that begins on a first date (e.g., Monday, Jan. 6, 2020) at a first time (e.g., 10:00 PM), that can be referred to as the current evening, when the cohort member first enters a bed with the intention of going to sleep (e.g., not if the cohort member intends to first watch television or play with a smart phone before going to sleep, etc.), and ends on a second date (e.g., Tuesday, Jan. 7, 2020) at a second time (e.g., 7:00 AM), that can be referred to as the next morning, when the cohort member first exits the bed with the intention of not going back to sleep that next morning.

In some implementations, the cohort member can manually define the beginning of a sleep session and/or manually terminate a sleep session. For example, the cohort member can select (e.g., by clicking or tapping) a user-selectable element that is displayed on the display device 172 of the user device 170 (FIG. 1) to manually initiate or terminate the sleep session.

The enter bed time t_{bed} is associated with the time that the cohort member initially enters the bed (e.g., bed 230 in FIG. 2) prior to falling asleep (e.g., when the cohort member lies down or sits in the bed). The enter bed time t_{bed} can be identified based on a bed threshold duration to distinguish between times when the cohort member enters the bed for sleep and when the cohort member enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the cohort member initially enters any location for sleeping (e.g., a couch, a chair, a sleeping bag, etc.).

The go-to-sleep time (GTS) is associated with the time that the cohort member initially attempts to fall asleep after entering the bed (t_{bed}). For example, after entering the bed, the cohort member may engage in one or more activities to wind down prior to trying to sleep (e.g., reading, watching TV, listening to music, using the user device 170, etc.). The initial sleep time (tₛₗₑₑₚ) is the time that the cohort member initially falls asleep. For example, the initial sleep time (tₛₗₑₑₚ) can be the time that the cohort member initially enters the first non-REM sleep stage.

The wake-up time t_{wake} is the time associated with the time when the cohort member wakes up without going back to sleep (e.g., as opposed to the cohort member waking up in the middle of the night and going back to sleep). The cohort member may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the cohort member goes back to sleep after each of the microawakenings MA₁ and MA₂. Similarly, the cohort member may have one or more conscious awakenings (e.g., awakening A) after initially falling asleep (e.g., getting up to go to the bathroom, attending to children or pets, sleep walking, etc.). However, the cohort member goes back to sleep after the awakening A. Thus, the wake-up time t_{wake} can be defined, for example, based on a wake threshold duration (e.g., the cohort member is awake for at least 15 minutes, at least 20 minutes, at least 30 minutes, at least 1 hour, etc.).

Similarly, the rising time t r i se is associated with the time when the cohort member exits the bed and stays out of the bed with the intent to end the sleep session (e.g., as opposed to the cohort member getting up during the night to go to the bathroom, to attend to children or pets, sleep walking, etc.). In other words, the rising time tᵣᵢₛₑ is the time when the cohort member last leaves the bed without returning to the bed until a next sleep session (e.g., the following evening). Thus, the rising time tᵣᵢₛₑ can be defined, for example, based on a rise threshold duration (e.g., the cohort member has left the bed for at least 15 minutes, at least 20 minutes, t_{bed} at least 30 minutes, at least 1 hour, etc.). The enter bed time t_{bed} time for a second, subsequent sleep session can also be defined based on a rise threshold duration (e.g., the cohort member has left the bed for at least 4 hours, at least 6 hours, at least 8 hours, at least 12 hours, etc.).

As described above, the cohort member may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the cohort member. For a standard cohort member who goes to bed in the evening, then wakes up and goes out of bed in the morning any period (between the cohort member waking up (t_{wake}) or raising up (tᵣᵢₛₑ), and the cohort member either going to bed (t_{bed}), going to sleep (t_{GTS}) or falling asleep (tₛₗₑₑₚ) of between about 12 and about 18 hours can be used. For cohort members that spend longer periods of time in bed, shorter threshold periods may be used (e.g., between about 8 hours and about 14 hours). The threshold period may be initially selected and/or later adjusted based on the system monitoring the cohort member's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 301 of FIG. 3, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the cohort member is initially falling asleep, the cohort member may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (twee). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

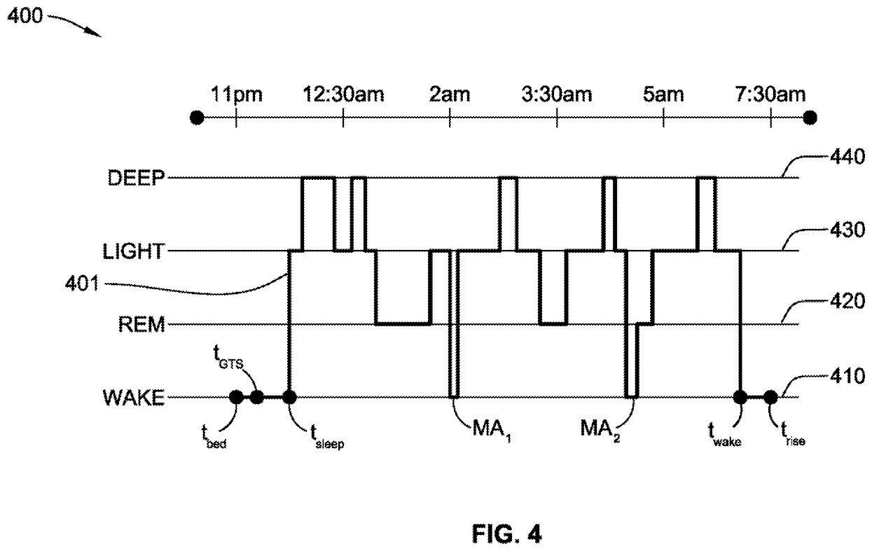

Referring to FIG. 4, an exemplary hypnogram 400 corresponding to the timeline 400 (FIG. 4), according to some implementations, is illustrated. As shown, the hypnogram 400 includes a sleep-wake signal 401, a wakefulness stage axis 410, a REM stage axis 420, a light sleep stage axis 430, and a deep sleep stage axis 440. The intersection between the sleep-wake signal 401 and one of the axes 410, 420, 430, 440 is indicative of the sleep stage at any given time during the sleep session.

The sleep-wake signal 401 can be generated based on physiological data associated with the cohort member (e.g., generated by one or more of the sensors 130 (FIG. 1) described herein). The sleep-wake signal can be indicative of one or more sleep states or stages, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 400 is shown in FIG. 4 as including the light sleep stage axis 430 and the deep sleep stage axis 440, in some implementations, the hypnogram 400 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 114.

The hypnogram 400 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the cohort member to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the cohort member is awake between the initial sleep time and the wake-up time. Thus, the wake-after-sleep onset includes short and micro-awakenings during the sleep session (e.g., the micro-awakenings MA₁ and MA₂ shown in FIG. 4), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the cohort member. For example, if the cohort member enters the bed and spends time engaged in other activities (e.g., watching TV) before sleep, the sleep efficiency will be reduced (e.g., the cohort member is penalized). In some implementations, the sleep efficiency (SE) can be calculated based on the total time in bed (TIB) and the total time that the cohort member is attempting to sleep. In such implementations, the total time that the cohort member is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the cohort member had two micro-awakenings (e.g., micro-awakening MA₁ and micro-awakening MA₂ shown in FIG. 4), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 130 can be used to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t wake), the rising time (tᵣᵢₛₑ), or any combination thereof, which in turn define the sleep session. For example, the enter bed time t_{bed} can be determined based on, for example, data generated by the motion sensor 138, the microphone 140, the camera 150, or any combination thereof. The go-to-sleep time can be determined based on, for example, data from the motion sensor 138 (e.g., data indicative of no movement by the cohort member), data from the camera 150 (e.g., data indicative of no movement by the cohort member and/or that the cohort member has turned off the lights), data from the microphone 140 (e.g., data indicative of the using turning off a TV), data from the user device 170 (e.g., data indicative of the cohort member no longer using the user device 170), data from the pressure sensor 132 and/or the flow rate sensor 134 (e.g., data indicative of the therapy user turning on the respiratory device 122, data indicative of the therapy user donning the user interface 124, etc.), or any combination thereof.

While hypnogram 400 depicts progressively shorter REM stages as the sleep session progresses, that is not always the case. In some cases, the duration of REM stages progressively increases as the sleep session progresses (e.g., with the first REM stage being shorter than the last REM stage).

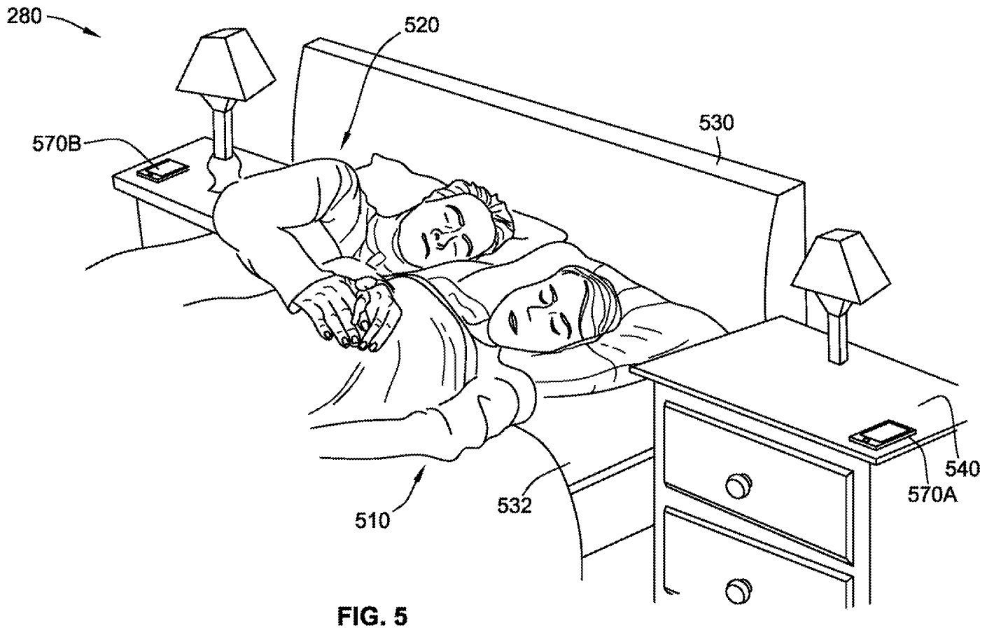

FIG. 5 is a perspective view of a pair of cohort members, including a first cohort member 510 and a second cohort member 520, according to certain aspects of the present disclosure. In some cases, aspects and features of the present disclosure can be used between two or more cohort members 510, 520 who do not make use of any respiratory therapy device or other sleep-related therapy device. In such cases, the system (e.g., system 100 of FIG. 1) can still monitor the cohort members 510, 520 and determine sleep performance metrics, which can be used to determine concerted sleep performance metrics, evaluate individual or cohort goals, generate individual or cohort coaching suggestions, or otherwise improve the sleep quality of the cohort members 510, 520.

Cohort member 510 and cohort member 520 are both sleeping in a bed 530 on a mattress 532. The system for tracking the sleep sessions of the cohort members 510, 520 can be implemented via a first user device 570A and a second user device 570B. User device 570A can be a smartphone associated with cohort member 510, and user device 570B can be a smartphone associated with cohort member 520, although other devices can be used such as a sonar-enabled and/or radar-enabled (optionally further comprising a microphone) bedside device configured to monitor physiological signals, such as cardiac, respiratory and/or motion signals.

A distance between the first user device 570A and the second user device 570B can be estimated based on sensor data collected by the one or more sensors within each of the user devices 570A, 570B. In some cases, the distance can be estimated based on signal strength of a wireless signal, such as a Bluetooth signal transmitted between the user devices 570A, 570B. In some cases, the distance can be estimated based on echoes detected by microphones of user devices 570A, 570B.

The distance between the user devices 570A, 570B can be used to infer whether or not cohort member 520 is sleeping in the same environment 500 as cohort member 510. For example, if the distance is determined to be relatively small, as seen in FIG. 5, an inference can be made that cohort member 520 is sleeping in the same bed as cohort member 510. At a slightly larger distance, an inference may be made that the cohort members 510, 520 are sleeping in the same room. In some cases, the distance can indicate that the cohort members 510, 520 are sleeping in adjacent room. In some cases, the distance can indicate that the cohort members 510, 520 are sleeping in the same house (e.g., same building). In some cases, the distance can indicate that cohort member 520 is not sleeping in the same environment 500 as cohort member 510.

In some cases, the distance between user devices 570A, 570B can be used to better identify location(s) of one or both of cohort members 510, 520, such as through echolocation or detection via other sensors. For example, knowledge of the distance between user devices 570A, 570B combined with knowledge of the distance between cohort member 510 and each of user devices 570A, 570B can be used to accurately locate cohort member 510 within the environment 500.

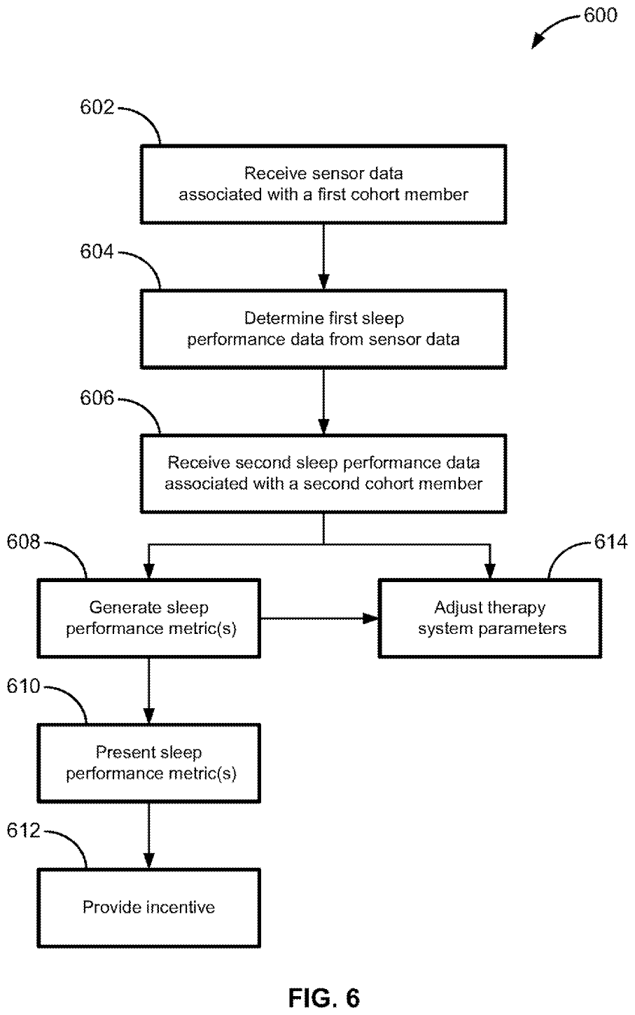

FIG. 6 is a flowchart depicting a process 600 for generating and presenting sleep performance metrics for a sleep cohort according to certain aspects of the present disclosure. Process 600 can be performed by system 100 of FIG. 1 or components thereof, or multiple instances or components of system 100 of FIG. 1.

At block 602, sensor data is received. The sensor data can be received from one or more sensors. The sensor data received at block 602 is associated with a sleep session of an individual in an environment. In other words, the sensor data received at block 602 includes sensor data obtained of the individual (e.g., a first cohort member, such as a therapy-adjacent individual) engaging in the sleep session. In some cases, the sensor data can also include sensor data obtained prior to or subsequent to the sleep session.

The environment can be a bed, a room, a set of adjacent rooms, or a house or other building. In some cases, the sensor data received at block 602 can also be associated with a sleep session of a second individual (e.g., a second cohort member, such as a therapy user) in the environment.

At block 604, the sensor data is used to determine first sleep performance data. The first sleep performance data includes data about the performance of the first cohort member's sleep session. Determining sleep performance data can include analyzing the sensor data to identify various metrics associated with the first cohort member's sleep session, such as sleep quality data. Sleep performance data can include sleep stage information, sleep state information, and/or sleep performance data, where appropriate.

At block 606, second sleep performance data can be received. The second sleep performance data is associated with a second cohort member, and more specifically with a sleep session of the second cohort member in the same environment as the first cohort member. The sleep session of the second cohort member can overlap, fully or partially, with the sleep session of the first cohort member.

In some cases, the second sleep performance data has already been determined from separate sensor data associated with the second cohort member by the time it is received at block 606. In some cases, receiving the second sleep performance data at block 606 can include determining the second sleep performance data from sensor data, such as the sensor data of block 602 or separate sensor data.

At block 608, one or more sleep performance metrics can be generated from the first sleep performance data and the second sleep performance data. A sleep performance metric can be any useful metric for measuring the performance of a sleep session. In some cases, the one or more sleep performance metrics include i) a concerted sleep performance score; ii) an individual sleep performance score associated with the first cohort member; iii) an individual sleep performance score associated with the second cohort member; iv) a hypnogram associated with the first cohort member; v) a hypnogram associated with the second cohort member; vi) a therapy score for a therapy user; vii) a resonance score; or viii) any combination of i-vii.

In some cases, the sleep performance metric can be a concerted sleep performance score that is calculated using a first sleep performance score associated with the first cohort member and a second sleep performance score associated with the second cohort member.

In some cases, generating sleep performance metric(s) at block 608 can include synchronizing first sensor data and second sensor data. The first sensor data can be the sensor data received at block 602 from a first set of one or more sensors associated with the first cohort member. The second sensor data can be sensor data received from a second set of one or more sensors associated with the second cohort member.

At block 610, the sleep performance metric(s) can be presented. Presentation at block 610 can include presenting one or more sleep performance metrics to the first cohort member, to the second cohort member, to a third party, or any combination thereof. Presentation can include presenting component and/or subcomponent scores associated with the sleep performance metric(s), and optionally an indication as to the level of contribution the component and/or subcomponent scores give to the given sleep performance metric.

In some cases, presenting sleep performance metrics at block 610 can include generating an entry on a feed associated with a cohort member or the cohort. The feed can be a social media feed or a similar feed. The feed can contain summary information, sleep performance metrics, or other such information. The feed can be interactive to permit other cohort members or third parties to interact with the entries on the feed and thus provide encouragement to the cohort member to improve their sleep quality.

In some cases, the first cohort member is a therapy-adjacent individual and the second cohort member is a therapy user, although in some cases the opposite can be true. In some cases, both the first cohort member and second cohort member are therapy users. When a cohort member is a therapy user, the sensor data can include data from one or more sensors associated with the therapy user's therapy device and the sleep performance data can include therapy data associated with use of the therapy device.

In an example case, process 600 can be performed by a user device (e.g., smartphone) of the first cohort member. The user device can receive sensor data from one or more sensor data of the user device and/or one or more sensors operatively coupled to the user device. The user device can then determine first sleep performance data from the sensor data. The user device can then receive second sleep performance data. In some cases, the user device can receive sensor data and use that sensor data to determine the second sleep performance data. In some cases, however, the user device can receive second sleep performance data that has already been determined from sensor data (e.g., on a user device of the second cohort member).

In another example case, process 600 can be performed by a sever (e.g., a cloud server), which can receive sensor data and/or sleep performance data from one or more user devices.

In some cases, at optional block 612, an incentive can be provided. Providing an incentive can include determining that a sleep performance metric has met a threshold or determining that a cohort member or cohort has achieved a goal (which can be set, tracked and/or reported upon according to e.g., process 700) or completed a threshold number of coaching suggestions (which can be set, tracked and/or reported upon according to e.g., process 800). Providing the incentive can include initiating a transfer of the incentive (e.g., monetary award, gift card, gift, or the like) to the cohort member or cohort associated with the incentive. In some cases, providing the incentive at block 612 can include providing individual incentives to the first cohort member and second cohort member i) for meeting their own threshold sleep performance metrics, achieving their own goal, and/or completing a threshold number of coaching suggestions; ii) for another cohort member meeting their respective threshold sleep performance metrics, achieving their respective goal, and/or completing a threshold number of coaching suggestions; or iii) any combination of i or ii.

In some cases, at optional block 614, parameters of a therapy device (e.g., a respiratory therapy device) can be adjusted based on sensor data from block 602. The therapy device can be used by the second cohort member. Thus, the parameters of the therapy device can be adjusted based on the sleep session data associated with a sleep session of the therapy-adjacent individual. In some cases, adjusting therapy parameters at block 614 can include dynamically adjusting the parameters during the sleep session of the therapy user, although that need not always be the case. In some cases, adjusting therapy parameters at block 614 occurs after receiving second sleep performance data at block 606. In some cases, adjusting therapy parameters at block 614 occurs after generating sleep performance metric(s) at block 608.

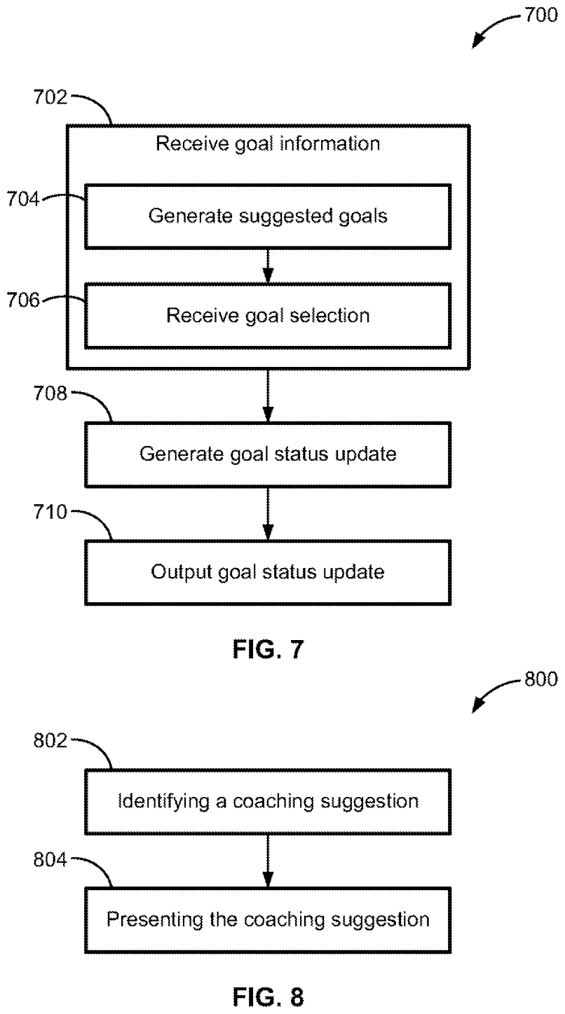

FIG. 7 is a flowchart depicting a process 700 for tracking goals for a sleep cohort according to certain aspects of the present disclosure. Process 700 can be performed by system 100 of FIG. 1 or components thereof, or multiple instances or components of system 100 of FIG. 1.

At block 702, goal information is received. Goal information can include information indicative of a goal and the goal's association with a sleep session of a first cohort member, with a sleep session of a second cohort member, with a cohort sleep session, or any combination thereof.

In some cases, goal information can include a target completion date. In some cases, the target completion date can be user-selected. In some cases, the target completion date can be automatically determined based on any combination of sleep performance data (e.g., current or historical) for one or more cohort members. An automatically determined target completion date can be automatically set for the goal or can be presented to the cohort member and set upon confirmation or selection by the cohort member.

In some cases, goal information can be received directly from user input. In some cases, goal information can be received based on one or more generated goals. At block 704, one or more suggested goals can be generated. At block 706, a goal selection can be received indicating one or more of the one or more suggested goals to use as a goal.

Generation of suggested goals at block 704 can be performed automatically in response to receiving responses to one or more prompts (e.g., a questionnaire), or in response to receipt of sleep performance data and/or generated sleep performance metrics. The responses can be used to generate the set of suggested goals.

In some cases, generation of suggested goals can include identifying factors influencing a historical sleep performance metric of a cohort member or a cohort, then determining a suggested action that can be taken to improve a future sleep performance metric (e.g., a future instance of the historical sleep performance metric). The set of suggested goals can then be generated based on the suggested actions.

In some cases, generation of suggested goals can include receiving demographic information about a cohort member and then generating the one or more suggested goals based on the demographic information. For example, if demographic information received about a cohort member is indicative that the cohort member may suffer from certain factors that may affect a sleep performance metric, one or more suggested goals can be generated based on the factors to improve the sleep performance metric. In some cases, generating one or more suggested goals based on demographic information can include accessing a database containing suggested goals associated with individuals sharing demographic information with the cohort member.

In some cases, generating suggested goals includes receiving historical therapy device usage information (e.g., historical respiratory therapy device usage) and generating suggested goal(s) using the received historical therapy usage information. Thus, generated goals can be tailored to a therapy user's past use of a therapy device (as determined from therapy device usage data, such as component data for generating a myAir™ score as described herein).

In some cases, generating suggested goals includes receiving subjective feedback associated with historical sleep sessions and generating suggested goal(s) using the subjective feedback. Thus, generated goals can be based on a cohort member's own subjective interpretation of previous sleep sessions.

At block 708, a goal status update can be generated. The goal status update can include information about the cohort member's, or the cohort's, progression towards meeting the goal. Generating the goal status update can include evaluating the goal using sleep performance data (e.g., the first sleep performance data and/or second sleep performance data from process 600 of FIG. 6). In some cases, evaluating the goal can include using sensor data. Evaluating the goal using sensor data can include determining sleep quality, determining a sleep-related metrics (e.g., a sleep performance metric), or determining other information using the sensor data. In an example, a goal based on distance between cohort members while they sleep can be evaluated using sensor data indicative of a distance between the cohort members (or the user devices of the cohort members).

At block 710, the goal status update can be output. Outputting the goal status update can include transmitting a status update to another computing device or presenting the goal status update on a display (e.g., a display of a user device). Other techniques can be used.

FIG. 8 is a flowchart depicting a process 800 for generating coaching suggestions for a sleep cohort according to certain aspects of the present disclosure. Process 800 can be performed by system 100 of FIG. 1 or components thereof, or multiple instances or components of system 100 of FIG. 1.

At block 802, a coaching suggestion can be identified. The coaching suggestion can be identified to improve a future sleep performance metric of a cohort member or a cohort.

In some cases, a coaching suggestion can be received directly from user input (e.g., from user input by another cohort member). In some cases, a coaching suggestion can be identified automatically in response to receiving responses (e.g., subjective feedback) to one or more prompts (e.g., a questionnaire). The responses can be used to generate the coaching suggestion.

In some cases, identification of coaching suggestions can include identifying factors influencing a historical sleep performance metric of a cohort member or a cohort, then determining a suggested action that can be taken to improve a future sleep performance metric (e.g., a future instance of the historical sleep performance metric). The coaching suggestion can then be generated based on the suggested actions.

At block 804, the coaching suggestion can be presented. Presenting the coaching suggestion can include transmitting the coaching suggestion to another computing device or presenting the coaching suggestion on a display (e.g., a display of a user device). Other techniques can be used.

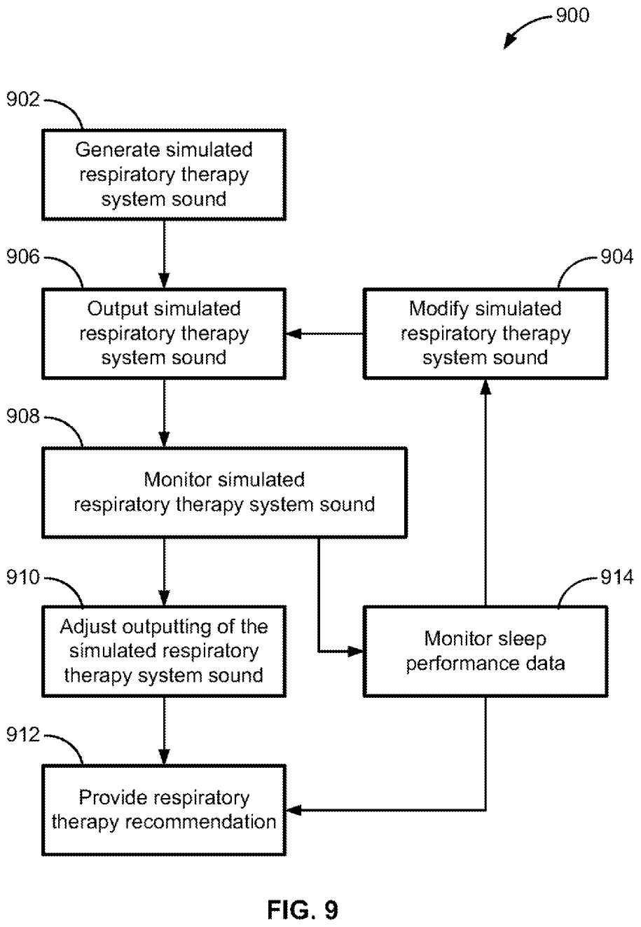

FIG. 9 is a flowchart depicting a process 900 for generating a simulated respiratory therapy device sound according to certain aspects of the present disclosure. Process 900 can be performed by system 100 of FIG. 1 or components thereof, or multiple instances or components of system 100 of FIG. 1.

At block 902, a simulated respiratory therapy device sound can be generated.

Generation of the simulated respiratory therapy device sound can include electronically generating the simulated respiratory therapy device sound or accessing a file containing a recording of a simulated respiratory therapy device sound. The simulated respiratory therapy device sound that is generated at block 902 can be based on a selected model of respiratory therapy device, selected attachments (e.g., user interface and/or conduit), and/or selected settings for the respiratory therapy device. In some cases, the simulated respiratory therapy device sound can be generated based on prescribed or actual settings for a future therapy user's or therapy user's respiratory therapy device.

At bock block 906, the simulated respiratory therapy device sound can be output. Outputting the simulated respiratory therapy device sound can include playing the simulated respiratory therapy device sound over a speaker. At block 908, the simulated respiratory therapy device sound can be monitored. Monitoring the simulated respiratory therapy device sound can include monitoring the simulated respiratory therapy device sound using a microphone.

At block 910, the simulated respiratory therapy device sound can be adjusted based on the monitored simulated respiratory therapy device sound. Adjusting the simulated respiratory therapy device sound based on the monitored simulated respiratory therapy device sound can include making adjustments to the volume or other characteristics of the simulated respiratory therapy device sound being output. In some cases, making adjustments at block 910 can include applying one or more filters to the simulated respiratory therapy device sound being output. Adjusting the simulated respiratory therapy device sound at block 910 can be performed to ensure the monitored simulated respiratory therapy device sound matches a desired or expected respiratory therapy device sound.

At optional block 912, a respiratory therapy recommendation can be made. The respiratory therapy recommendation can include a recommendation for a particular respiratory therapy device model, a particular conduit model or type, a particular user interface model or type, one or more settings for use on the respiratory therapy device, or any combination thereof. In some cases, providing the respiratory therapy recommendation can include receiving an adjustment command to adjust a volume of the simulated respiratory therapy device sound. In such cases, the respiratory therapy recommendation is based on the adjusted volume of the simulated respiratory therapy device sound.

At optional block 914, sleep performance data can be monitored for a cohort member or a cohort. The monitored sleep performance data can be used to determine sleep performance metrics for the cohort member or cohort. In some cases, monitoring sleep performance data at block 914 can include using the sleep performance data to modify the simulated respiratory therapy device sound at optional block 904. In some cases, monitoring sleep performance data at block 914 can include using the sleep performance data to inform a respiratory therapy recommendation at block 912.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art. Numerous changes to the disclosed embodiments can be made in accordance with the disclosure herein, without departing from the spirit or scope of the present disclosure. Thus, the breadth and scope of the present disclosure should not be limited by any of the above described embodiments.

Although certain aspects of the present disclosure have been illustrated and described with respect to one or more implementations, equivalent alterations and modifications will occur or be known to others skilled in the art upon the reading and understanding of this specification and the annexed drawings. In addition, while a particular feature of an aspect of the present disclosure may have been disclosed with respect to only one of several implementations, such feature may be combined with one or more other features of the other implementations as may be desired and advantageous for any given or particular application.

One or more elements or aspects or steps, or any portion(s) thereof, from one or more of any of claims 1 to 87 below can be combined with one or more elements or aspects or steps, or any portion(s) thereof, from one or more of any of the other claims 1 to 87 or combinations thereof, to form one or more additional implementations and/or claims of the present disclosure.

What is claimed is:

1. A method comprising:
receiving sensor data from one or more sensors, the sensor data being associated with a sleep session of an individual in an environment;
determining first sleep performance data from the sensor data;
receiving second sleep performance data, the second sleep performance data being associated with a sleep session of a user of a respiratory therapy device in the environment;
generating one or more sleep performance metrics using the first sleep performance data and the second sleep performance data; and
presenting the one or more sleep performance metrics.

2. The method of claim 1, wherein the one or more sleep performance metrics includes i) a concerted sleep performance score; ii) an individual sleep performance score associated with the individual; iii) an individual sleep performance score associated with the user; iv) a hypnogram associated with the individual; v) a hypnogram associated with the user; vi) a therapy score for the user; vii) a resonance score; or viii) any combination of i-vii.

3. The method of claim 1 or claim 2, wherein the sleep session of the individual and the sleep session of the user overlap in time.

4. The method of any one of claims 1 to 3, wherein the first sleep performance data includes sleep stage information or sleep state information; and wherein the second sleep performance data includes respiratory therapy device usage information.

5. The method of any one of claims 1 to 4, further comprising:
receiving goal information associated with the first user and the second user, wherein the goal information is indicative of a goal associated with i) the sleep session of the individual, ii) the sleep session of the user, or iii) a combination of i and ii;
generating a goal status update, wherein generating the goal status update includes evaluating the goal information using i) the first sleep performance data; ii) the second sleep performance data; or iii) a combination of i and ii; and
outputting the goal status update.

6. The method of claim 5, wherein receiving goal information includes:
generating a set of one or more suggested goals; and
receiving a selection for a selected goal out of the set of suggested goals.

7. The method of claim 6, wherein generating the set of suggested goals includes:
presenting a questionnaire containing one or more questions;
receiving response information in response to presenting the questionnaire; and
generating the set of suggested goals using the received response information.

8. The method of claim 6 or claim 7, wherein generating the set of suggested goals includes:
accessing historical sleep performance data associated with historical sleep performance metrics;
identifying one or more factors as influencing the historical sleep performance metrics;
determining, for each of the one or more factors, a suggested action estimated to improve a future sleep performance metric; and
generating the set of suggested goals using the suggested action for each of the one or more factors.

9. The method of any one of claims 6 to 8, wherein generating the set of suggested goals includes:
receiving demographic information associated with the individual or the user; and
generating the set of suggested goals using the received demographic information.

10. The method of any one of claims 6 to 9, wherein generating the set of suggested goals includes:
receiving historical respiratory therapy device usage information associated with the user; and
generating the set of suggested goals using the received historical respiratory therapy device usage information.

11. The method of any one of claims 6 to 10, wherein generating the set of suggested goals includes:
receiving subjective feedback associated with a plurality of historical sleep sessions; and
generating the set of suggested goals using the subjective feedback.

12. The method of any one of claims 5 to 11, wherein evaluating the goal further includes using the sensor data.

13. The method of claim 12, wherein evaluating the goal using the sensor data includes estimating a distance between the individual and the user using the sensor data.

14. The method of any one of claims 5 to 13, wherein receiving goal information includes receiving a target completion date associated with the goal, and wherein receiving the target completion data includes automatically determining the target completion date using i) the first sleep performance data; ii) the second sleep performance data; iii) historical sleep performance data; or iv) any combination of i-iii.

15. The method of any one of claims 5 to 14, wherein the goal information includes a goal associated with a start time of a future sleep session of the individual and a start time of a future sleep session of the user.

16. The method of any one of claims 5 to 15, wherein the goal information includes a goal associated with a distance between the individual and the user at a future sleep session.

17. The method of any one of claims 5 to 16, wherein the goal information includes a goal associated with a future use of the respiratory therapy device.

18. The method of any one of claims 5 to 17, wherein evaluating the goal information includes determining that the goal is completed, wherein the goal status update is indicative of the goal being completed, and wherein the method further comprises:
determining, in response to determining that the goal is completed, one or more suggested subsequent goals based at least in part on i) the completed goal; ii) a time taken to achieve the completed goal; iii) sleep performance data; iv) subjective data of the user; or v) any combination of i-iv; and
presenting the one or more suggested subsequent goals.

19. The method of claim 18, further comprising:
receiving a subsequent goal selection in response to presenting the one or more suggested subsequent goals, the subsequent goal selection indicative of a subsequent goal out of the one or more suggested subsequent goal;
receiving subsequent goal information associated with the subsequent goal;
generating a subsequent goal status update, wherein generating the subsequent goal status update includes evaluating the subsequent goal information; and
outputting the subsequent goal status update.

20. The method of any one of claims 1 to 19, wherein generating the one or more sleep performance metrics includes generating a concerted sleep performance score, and wherein generating the concerted sleep performance score includes:
generating a first sleep performance score using the first sleep performance data;
generating a second sleep performance score using the second sleep performance data; and
generating the concerted sleep performance score using the first sleep performance score and the second sleep performance score.

21. The method of any one of claims 1 to 20, further comprising:
identifying a coaching suggestion for improving a future sleep performance metric; and
providing, after the first sleep session, the coaching suggestion.

22. The method of claim 21, wherein identifying the coaching suggestion includes:
receiving subjective feedback associated with a plurality of historical sleep sessions; and
generating the coaching suggestion using the subjective feedback.

23. The method of claim 21 or 22, wherein identifying the coaching suggestion includes:
accessing historical sleep performance data associated with historical sleep performance metrics;
identifying one or more factors as influencing the historical sleep performance metrics;
determining, for each of the one or more factors, a suggested action estimated to improve a future sleep performance metric; and
generating the coaching suggestion using the suggested change for each of the one or more factors.

24. The method of any one of claims 1 to 23, further comprising providing an incentive based on the first sleep performance data and the second sleep performance data.

25. The method of claim 24, wherein providing the incentive is further based on a comparison between the one or more sleep performance metrics and a historical sleep performance metric.

26. The method of claim 24 or claim 25, wherein generating the one or more sleep performance metrics includes generating a concerted sleep performance score, and wherein generating the concerted sleep performance score includes:
generating a first sleep performance score using the first sleep performance data;
generating a second sleep performance score using the second sleep performance data; and
generating the concerted sleep performance score using the first sleep performance score and the second sleep performance score; and
wherein providing the incentive occurs when the first sleep performance score exceeds a first threshold and the second sleep performance score exceeds a second threshold.

27. The method of claim 26, wherein providing the incentive includes providing a first individual incentive associated with the individual and providing a second individual incentive associated with the user.

28. The method of claim 26, further comprising:
providing a first individual incentive associated with the individual when the first sleep performance score exceeds the first threshold; and
providing a second individual incentive associated with the user when the second sleep performance score exceeds the second threshold.

29. The method of claim 26, further comprising:
providing a first individual incentive associated with the user when the first sleep performance score exceeds the first threshold; and
providing a second individual incentive associated with the individual when the second sleep performance score exceeds the second threshold.

30. The method of any one of claims 1 to 29, further comprising:
transmitting summary information based on the first sleep performance data, wherein the summary information, when received by a user device associated with the user, is usable to generate an entry on a feed of historical summary information associated with the individual.

31. The method of claim 30, further comprising:
receiving feedback in response to generation of the entry, wherein the feedback is indicative of a reaction.

32. The method of claim 30 or claim 31, wherein generating the one or more sleep performance metrics includes generating a first sleep performance score using the first sleep performance data, and wherein the summary information includes the first sleep performance score.

33. The method of any one of claims 1 to 32, further comprising:
receiving summary information on a user device associated with the individual, wherein the summary information is based on the second sleep performance data; and
generating an entry on a feed of historical summary information associated with the user using the received summary information.

34. The method of claim 33, wherein generating the one or more sleep performance metrics includes generating a second sleep performance score using the second sleep performance data, and wherein the summary information includes the second sleep performance score.

35. The method of any one of claims 1 to 34, wherein the second sleep performance data is determined using the sensor data, and wherein the sensor data is further associated with the sleep session of the user in the environment.

36. The method of any one of claims 1 to 35, wherein the second sleep performance data is determined using second sensor data from a second set of one or more sensors, the second sensor data being associated with the sleep session of the user in the environment.

37. A method, comprising:
receiving data associated with air supplied to a user interface using a respiratory therapy device, the user interface being worn by a user engaging in a sleep session in an environment;
receiving sleep session data associated with a sleep session of an individual in the environment, the individual being different than the user; and
determining an adjustment of a parameter of the respiratory therapy device in response to the received sleep session data.

38. The method of claim 37, wherein determining the adjustment of the parameter occurs dynamically during the sleep session of the user and the sleep session of the individual.

39. The method of claim 37 or claim 38, wherein the sleep session data includes sleep stage data of the individual, and wherein determining the adjustment of the parameter of the respiratory therapy device is based on the sleep stage data.

40. The method of any one of claims 37 to 39, wherein determining the adjustment of the parameter of the respiratory therapy device includes:
determining a first setting for the parameter when the sleep session data is indicative that the individual is awake; and
determining a second setting for the parameter when the sleep session data is indicative that the individual is asleep, wherein the respiratory therapy device is quieter when the first setting of the parameter is used than when the second setting of the parameter is used.

41. The method of any one of claims 37 to 40, further comprising:
receiving first sensor data associated with the sleep session of the user;
receiving second sensor data associated with the sleep session of the individual, wherein the sleep session data associated with the second sleep session is determined using the second sensor data; and
synchronizing the first sensor data and the second sensor data.

42. The method of claim 41, further comprising improving a signal-to-noise ratio of a signal of the first sensor data using the synchronized second sensor data.

43. The method of claim 41 or claim 42, further comprising:
detecting a possible event using the first sensor data; and
confirming the event using the synchronized second sensor data.

44. The method of any one of claims 41 to 43, further comprising estimating a position of the user using the synchronized first sensor data and synchronized second sensor data.

45. The method of any one of claims 37 to 44, further comprising:
establishing a wireless connection with a user device associated with the individual, wherein receiving the sleep session data occurs using the wireless connection; and
measuring characteristics of the wireless connection; and
determining location information of the individual based on the measured characteristics of the wireless connection.

46. The method of claim 45, further comprising determining the adjustment to the parameter of the respiratory therapy device based on the location information.

47. The method of claim 45, wherein the wireless connection is a Bluetooth connection.

48. The method of any one of claims 1 to 47, wherein the environment is a building.

49. The method of any one of claims 1 to 47, wherein the environment is a pair of adjacent rooms.

50. The method of any one of claims 1 to 47, wherein the environment is a room.

51. The method of any one of claims 1 to 47, wherein the environment is a sleeping surface.

52. A system, comprising:
a respiratory therapy device for supplying air;
a user interface fluidly coupled to the respiratory therapy device to direct the supplied air to a user;
a control system including one or more processors; and
a memory coupled to the control system, the memory containing instructions which, when executed on the one or more processors, cause the one or more processors to perform operations including:
receiving data associated with the respiratory therapy device supplying air to the user interface while the user interface is worn by the user engaging in a sleep session in an environment;
receiving sleep session data associated with a sleep session of an individual in the environment, the individual being different than the user; and
determining an adjustment of a parameter of the respiratory therapy device in response to the received sleep session data.

53. The system of claim 52, wherein determining the adjustment of the parameter occurs dynamically during the sleep session of the user and the sleep session of the individual.

54. The system of claim 52 or claim 53, wherein the sleep session data includes sleep stage data of the individual, and wherein determining the adjustment of the parameter of the respiratory therapy device is based on the sleep stage data.

55. The system of any one of claims 52 to 54, wherein determining the adjustment of the parameter of the respiratory therapy device includes:
determining a first setting for the parameter when the sleep session data is indicative that the individual is awake; and
determining a second setting for the parameter when the sleep session data is indicative that the individual is asleep, wherein the respiratory therapy device is quieter when the first setting of the parameter is used than when the second setting of the parameter is used.

56. The system of any one of claims 52 to 55, wherein the operations further comprise:
receiving first sensor data associated with the sleep session of the user;
receiving second sensor data associated with the sleep session of the individual, wherein the sleep session data associated with the second sleep session is determined using the second sensor data; and
synchronizing the first sensor data and the second sensor data.

57. The system of claim 56, wherein the operations further comprise improving a signal-to-noise ratio of a signal of the first sensor data using the synchronized second sensor data.

58. The system of claim 56 or claim 57, wherein the operations further comprise:
detecting a possible event using the first sensor data; and
confirming the event using the synchronized second sensor data.

59. The system of any one of claims 56 to 58, wherein the operations further comprise estimating a position of the user using the synchronized first sensor data and synchronized second sensor data.

60. The system of any one of claims 52 to 59, wherein the operations further comprise:
establishing a wireless connection with a user device associated with the individual, wherein receiving the sleep session data occurs using the wireless connection; and
measuring characteristics of the wireless connection; and
determining location information of the individual based on the measured characteristics of the wireless connection.

61. The system of claim 60, wherein the operations further comprise determining the adjustment to the parameter of the respiratory therapy device based on the location information.

62. The system of claim 60, wherein the wireless connection is a Bluetooth connection.

63. The system of any one of claims 52 to 62, wherein the environment is a building.

64. The system of any one of claims 52 to 62, wherein the environment is a pair of adjacent rooms.

65. The system of any one of claims 52 to 62, wherein the environment is a room.

66. The system of any one of claims 52 to 62, wherein the environment is a sleeping surface.

67. A method, comprising:
generating a simulated respiratory therapy device sound;
outputting the simulated respiratory therapy device sound;
monitoring the outputted simulated respiratory therapy device sound using a microphone; and
adjusting output of the simulated respiratory therapy device sound based on the monitored outputted simulated respiratory therapy device sound.

68. The method of claim 67, further comprising accessing a set of prescribed respiratory therapy settings, wherein generating the simulated respiratory therapy device sound is based on the set of prescribed respiratory therapy settings.

69. The method of claim 67 or claim 68, further comprising accessing a set of therapy settings of a respiratory therapy device, wherein generating the simulated respiratory therapy device sound is based on the set of therapy settings of the respiratory therapy device.

70. The method of any one of claims 67 to 69, further comprising:
receiving an adjustment command;
adjusting volume of the simulated respiratory therapy device in response to receiving the adjustment command; and
providing a respiratory therapy recommendation based on the adjusted volume of the simulated respiratory therapy device.

71. The method of claim 70, wherein the respiratory therapy recommendation includes i) a respiratory therapy device model; ii) a user interface type; iii) a user interface model; iv) a conduit type; v) a conduit model; or vi) any combination of i-v.

72. The method of any one of claims 67 to 71, further comprising:
receiving sensor data from one or more sensors, wherein the sensor data is associated with a user engaging in a sleep session, wherein outputting of the simulated respiratory therapy device sound occurs during the sleep session;
determining sleep performance information using the sensor data; and
outputting the sleep performance information.

73. The method of claim 72, wherein the sensor data is associated with the user engaging in the sleep session in an environment, wherein the method further comprises:
receiving additional sleep performance information, the additional sleep performance information being associated with a sleep session of an individual in the environment, the individual being different than the user, wherein outputting of the simulated respiratory therapy device sound occurs during the sleep session of the individual; and
outputting the additional sleep performance information.

74. The method of claim 73, wherein receiving the additional sleep performance information includes:
receiving additional sensor data from the one or more sensors, wherein the additional sensor data is associated with the sleep session of the individual; and
determining the additional sleep performance information using the additional sensor data.

75. The method of claim 73, wherein receiving the additional sleep performance information includes:
receiving additional sensor data from one or more additional sensors, wherein the additional sensor data is associated with the sleep session of the individual, and wherein the one or more additional sensors are different than the one or more sensors; and
determining the additional sleep performance information using the additional sensor data.

76. The method of any one of claims 73 to 75, further comprising modifying output of the simulated respiratory therapy device sound during the sleep session, wherein modifying output of the simulated respiratory therapy device sound is based at least in part on the determined sleep performance information and the received additional sleep performance information.

77. The method of any one of claims 72 to 75, further comprising modifying output of the simulated respiratory therapy device sound during the sleep session.

78. The method of claim 77, wherein modifying output of the simulated respiratory therapy device sound is based on the determined sleep performance information.

79. The method of any one of claims 72 to 78, wherein generating the simulated respiratory therapy device sound is associated with a first respiratory therapy device model, the method further comprising:
retrieving historical sleep performance information associated with a historical sleep session, wherein the historical sleep session occurred during outputting of additional simulated respiratory device sound, wherein the additional simulated respiratory device sound is associated with a second respiratory therapy device model; and
generating a comparison between the sleep performance information and the historical sleep performance information.

80. The method of claim 79, further comprising generating a recommendation for the first respiratory therapy device model or the second respiratory therapy device model based on the generated comparison.

81. The method of any one of claims 67 to 80, further comprising:
receiving medical information associated with an individual; and
modifying output of the simulated respiratory therapy device sound based on the received medical information.

82. A system comprising:
a control system including one or more processors; and
a memory having stored thereon machine readable instructions;
wherein the control system is coupled to the memory, and the method of any one of claims 1 to 51 or claims 67 to 81 is implemented when the machine executable instructions in the memory are executed by at least one of the one or more processors of the control system.

83. A system for shared sleep scoring, the system including a control system configured to implement the method of any one of claims 1 to 36 or claims 48 to 51.

84. A system for controlling respiratory therapy, the system including a control system configured to implement the method of any one of claims 37 to 51.

85. A system for simulating respiratory therapy, the system including a control system configured to implement the method of any one of claims 67 to 81.

86. A computer program product comprising instructions which, when executed by a computer, cause the computer to carry out the method of any one of claims 1 to 51 or claims 67 to 81.

87. The computer program product of claim 86, wherein the computer program product is a non-transitory computer readable medium.
